(19) United States
(12) Patent Application Publication
(10) Pub. No.: US 2025/0271428 A1
(43) Pub. Date: Aug. 28, 2025

(54) PEPTIDE-BASED BIOMARKERS AND RELATED ASPECTS FOR DISEASE DETECTION

(71) Applicant: ARIZONA BOARD OF REGENTS ON BEHALF OF ARIZONA STATE UNIVERSITY, Scottsdale, AZ (US)

(72) Inventors: Laimonas KELBAUSKAS, Gilbert, AZ (US); Neal WOODBURY, Tempe, AZ (US); Visar BERISHA, Tempe, AZ (US); Joseph LEGUTKI, Chandler, AZ (US); Pradyumna KADAMBI, Phoenix, AZ (US)

(73) Assignee: ARIZONA BOARD OF REGENTS ON BEHALF OF ARIZONA STATE UNIVERSITY, Scottsdale, AZ (US)

(21) Appl. No.: 18/878,826

(22) PCT Filed: Jun. 29, 2023
(86) PCT No.: PCT/US2023/026611

§ 371 Date: Dec. 24, 2024

Related U.S. Application Data
(60) Provisional application No. 63/358,023, filed on Jul. 1, 2022.

(51) Int. Cl. G01N 33/569; A61K 31/43; A61K 31/545; A61K 31/546; A61K 31/65; A61K 31/7048; A61K 31/7052; G16B 40/00

(52) U.S. Cl. CPC G01N 33/56911; A61K 31/43; A61K 31/545; A61K 31/546; A61K 31/65; A61K 31/7048; A61K 31/7052; G16B 40/00; G01N 2333/20; G01N 2469/10

(57) ABSTRACT

Biomarkers and machine learning techniques to identify biomarkers are disclosed herein. In one particular implementation, the present disclosure relates to the identification of biomarkers to be used in the detection and diagnosis of LD. In particular, the present disclosure relates to machine learning-based techniques for the discovery of biomarkers for detecting and diagnosing LD and the use of those biomarkers. In particular, the disclosure describes short sequences of amino acids (i.e., peptides) and proteins from the B. burgdorferi proteome that can be used for detection of Lyme disease in patient samples.

### CROSS-REFERENCE TO RELATED APPLICATIONS

This application claims the benefit of and priority to U.S. Provisional Patent Application Ser. No. 63/358,023, filed Jul. 1, 2022, the disclosure of which is incorporated herein by reference.

### STATEMENT OF GOVERNMENT SUPPORT

This invention was made with government support under R43 AI162473 awarded by the National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 28, 2023, is named “0391.0051-PCT.xml” and is 147 kilobytes in size. The sequence listing contained in this .XML file is part of the specification and is hereby incorporated by reference herein in its entirety.

### BACKGROUND

Among other tick-borne diseases. Lyme disease (LD) represents one of the most prominent challenges due to the lack of reliable early diagnostic tools and targeted treatment options. Existing clinical diagnostic assays for LD are based on a two-tier combination of tests that involve ELISA and immunoblotting approaches targeting several well-known immunogenic proteins from the Borrelia burgdorferi (B. burgdorferi) proteome. Despite their widespread use, current tests can produce high false negative rates. Furthermore, detection of chronic Lyme disease (CLD), a subtype of LD that develops in estimated 10-20% of LD patients after primary treatment with an antibiotics regimen, is even more troublesome due to the inability to detect specific immune system response with the conventional molecular assays. In such cases, CLD is diagnosed solely based on clinical disease manifestations. However, the clinical symptoms characteristic to CLD overlap with other illnesses, such as depression or fibromyalgia, making the clinical utility of this approach in terms of both diagnostics and treatment highly complex.

### SUMMARY

The present disclosure generally relates to the use of machine learning techniques to identify biomarkers that can be used to detect and diagnose a disease, including tick-borne diseases, such as Lyme disease (LD). These and other aspects will be apparent upon a complete review of the present disclosure, including the accompanying figures.

In one aspect, the present disclosure relates to a method of detecting Lyme disease in a subject. The method includes detecting a presence of one or more of the B. burgdorferi antigenic peptides or proteins listed in TABLE 7, 8, 9, 10, and/or 11 in a sample obtained from the subject, thereby detecting Lyme disease in the subject. In some embodiments, the B. burgdorferi antigenic peptides are selected from IIYRKNEEFI (SEQ ID NO: 36). IFNKKDNVVY (SEQ ID NO: 37), KKFIIDHTKE (SEQ ID NO: 38), IKLIKDIHKD (SEQ ID NO: 39), and KNFIKDVLKD (SEQ ID NO: 40). In some embodiments, the method includes detecting a presence of one or more amino acids that encode one or more of the B. burgdorferi antigenic peptides or proteins listed in TABLE 7, 8, 9, 10, and/or 11 in a sample obtained from the subject. In some embodiments, detecting the presence of the one or more of the antigenic peptides or proteins from the pathogen (B. burgdorferi in LD) proteome listed in TABLE 7, 8, 9, 10, and/or 11 comprises detecting a presence of one or more antibodies in the sample that bind to the one or more antigenic peptides or B. burgdorferi proteins listed in TABLE 7, 8, 9, 10, and/or 11. In some embodiments, the method includes detecting the presence of the one or more of the B. burgdorferi antigenic peptides or proteins listed in TABLE 7, 8, 9, 10, and/or 11 comprises the use of antibodies raised against the one or more of the B. burgdorferi antigenic peptides or proteins listed in TABLE 7, 8, 9, 10, and/or 11 in the sample.

In one aspect, the present disclosure relates to a method of detecting Lyme disease in a subject. The method includes detecting a presence of one or more nucleic acids that encode one or more of the B. burgdorferi antigenic peptides or proteins listed in TABLE 7, 8, 9, 10, and/or 11 in a sample obtained from the subject, thereby detecting Lyme disease in the subject. In some embodiments, detecting the presence of the one or more nucleic acids that encode the one or more B. burgdorferi antigenic peptides or proteins listed in TABLE 7, 8, 9, 10, and/or 11 comprises sequencing the one or more nucleic acids in the sample.

In some embodiments, the method further comprises obtaining the sample from the subject. In some embodiments, the method further comprises administering at least one therapeutic treatment to the subject. In some embodiments, administering the at least one therapeutic treatment comprises administering an effective amount of an antibiotic selected from oxytetracycline, doxycycline, minocycline, amoxicillin, penicillin, cefaclor, cefbuperazone, cefminox, cefotaxime, cefotetan, cefmetazole, cefoxitin, cefuroxime axetil, cefuroxime acetyl, ceftin, ceftriaxone, azithromycin, clarithromycin, erythromycin, and combination thereof. Some embodiments provide reaction mixtures that comprise reagents for performing the methods disclosed herein. Some embodiments provide kits that comprise reagents for performing the methods disclosed herein.

In another aspect, the present disclosure provides a computer-implemented method of generating predicted binding intensities from a microarray peptide data set. The method includes passing the microarray peptide data set through an electronic neural network model, wherein the microarray peptide data set is obtained from a microarray that comprises a quasi-random set of peptides using one or more antibodies or donor serum sample and wherein the electronic neural network model has been trained to predict binding intensities of peptides not present on the microarray. The method also includes outputting from the electronic neural network the predicted binding intensities of peptides not represented on the microarray. In some embodiments, the binding intensities associated with the microarray peptide data set comprise binding intensities with one or more proteomes selected from the group consisting of: a proteome associated with a vector of a disease, a proteome associated with a carrier of a vector of a disease, and a human proteome. In some embodiments, the computer-implemented method further comprises passing the predicted binding intensities from the microarray peptide data set to one or more classifiers that have been trained using one or more potential biomarkers to distinguish between a disease state and a non-disease state. In some embodiments, predicted strong binding targets in the proteome(s) are used to identify immunogenic full proteins that can further be used as biomarkers in orthogonal assays. In some embodiments, the method further includes passing the predicted binding intensities of peptides that are not present on the microarray set to one or more classifiers that have been trained using one or more potential biomarkers to distinguish between a disease state and a non-disease state.

In some embodiments, the computer-implemented method further comprises mapping, using an electronic neural network amino language model, the microarray peptide data set to a set of embeddings, and passing the set of embeddings to a machine learning model to determine the predicted binding intensities of a peptides not represented on the peptide microarray. Such peptides can represent tiled entire proteomes of pathogens, disease vectors, human or other organisms. Such peptides could also be randomly generated to enable for discovery of additional, possibly more potent biomarkers. In some embodiments, the computer-implemented method further comprises ranking at least a subset of the new set of peptides that are not contained on the microarrays based upon the predicted binding intensities from the microarray peptide data set to produce a set of ranked peptides, producing a classification model using the set of ranked peptides, which classification model classifies a sample from a test subject as being positive or negative for a disease, assessing a performance of the classification model to produce a classification model performance assessment measure, and determining whether the set of ranked peptides comprises candidate biomarkers for detecting a presence of the disease in test subjects based on the classification model performance assessment measure. In some embodiments, the disease is Lyme disease.

In some embodiments, the computer-implemented method includes ranking at least a subset of a set of peptides not represented on the array based upon the predicted binding intensities obtained using a machine learning model trained on the microarray peptide data set to produce a set of ranked peptides; using statistical methods, identifying protein biomarkers from proteomes of the pathogen and/or other associated organisms; producing a classification model using predicted intensity values of the set of ranked peptides that are not represented on the microarray, which classification model classifies a sample from a test subject as being positive or negative for a disease; assessing a performance of the classification model to produce a classification model performance assessment measure; and determining whether the set of ranked peptides comprises candidate biomarkers for detecting a presence of the disease in test subjects based on the classification model performance assessment measure.

In some embodiments, the classification model is selected from the group consisting of: a general linear model, a support vector machine, an extreme gradient boosting model, an electronic neural network model, or combinations thereof. In some embodiments, the disease is associated with a pathogen and a carrier and wherein the method further comprises filtering, from the set of carrier-related peptides, wherein the carrier-related peptides are associated with other pathogens associated with the carrier. In some embodiments, the pathogen is Borrelia burgdorferi and the carrier is the blacklegged tick Ixodes scapularis. In some embodiments, the set of the peptides of interest not represented on the microarray is ranked according to p-values associated with corresponding predicted binding intensities. In some embodiments, the set of peptides corresponds to a set of n-highest ranked peptides, wherein n is an integer greater than one. In some embodiments, assessing the performance of the classification model comprises generating a ROC curve corresponding to the performance of the classification model.

In another aspect, the present disclosure relates to a system for generating predicted binding intensities from a microarray peptide data set using an electronic neural network. The system includes a processor, and a memory communicatively coupled to the processor, the memory storing instructions which, when executed on the processor, perform operations comprising: passing the microarray peptide data set through the electronic neural network, wherein the microarray peptide data set is obtained from a microarray that comprises a quasi-random set of peptides using one or more antibodies or donor serum sample and wherein the electronic neural network has been trained to predict binding intensities associated with the microarray peptide data set, and outputting from the electronic neural network the predicted binding intensities of another set of peptides not represented on the microarray. In some embodiments, the instructions which, when executed on the processor, further perform operations comprising: passing the predicted binding intensities to a new set of peptides to one or more classifiers that have been trained using one or more potential biomarkers to distinguish between a disease state and a non-disease state. In some embodiments, the instructions which, when executed on the processor, further perform operations comprising: mapping, using an electronic neural network amino language model, the microarray peptide data set to a set of embeddings, and passing the set of embeddings to a machine learning model to determine the predicted binding intensities from the microarray peptide data set. In some embodiments, the instructions which, when executed on the processor, further perform operations comprising: ranking a new set of peptides not represented on the microarray to produce a set of ranked peptides, producing a classification model using the set of ranked peptides, which classification model classifies a sample from a test subject as being positive or negative for a disease, assessing a performance of the classification model to produce a classification model performance assessment measure, and determining whether the set of ranked peptides comprises candidate biomarkers for detecting a presence of the disease in test subjects based on the classification model performance assessment measure.

### FIGURES

The accompanying drawings, which are incorporated in and form a part of the specification, illustrate the embodiments of the invention and together with the written description serve to explain the principles, characteristics, and features of the invention. In the drawings:

FIG. 1 depicts a flow diagram of a process for developing a disease predictive model in accordance with an embodiment.

FIG. 2A depicts a ROC curve for a classifier developed to distinguish between clinically confirmed LD cases and controls using anti-IgG secondary antibody in accordance with an embodiment.

FIG. 2B depicts a ROC curve for a classifier developed to distinguish between confirmed LD cases and controls using anti-IgM secondary antibody in accordance with an embodiment.

FIG. 2C depicts a ROC curve for a classifier developed to distinguish between clinically diagnosed, seronegative LD cases and controls using anti-IgG secondary antibody in accordance with an embodiment.

FIG. 3A depicts a graph showing the correlation between predicted and measured classifications by a trained machine learning model in accordance with an embodiment.

FIG. 3B depicts a comparison of the predictive binding intensity to the C6 peptide GKFAVKDGEK (SEQ ID NO: 126) of the V1sE protein for confirmed LD cases and controls in accordance with an embodiment.

FIG. 4A depicts a ROC curve for a classifier developed to distinguish between acute LD cases and controls in accordance with an embodiment.

FIG. 4B depicts a ROC curve for a classifier developed to distinguish between acute LD cases and controls in accordance with an embodiment.

FIG. 4C depicts a ROC curve for a classifier developed to distinguish between clinically diagnosed, seronegative LD cases and controls in accordance with an embodiment.

FIG. 5 depicts a ROC curve for a classifier developed to distinguish between acute confirmed and clinically diagnosed, seronegative LD cases and controls in accordance with an embodiment.

FIG. 6 are plots showing a comparison of binding (fluorescence) intensity distributions of 2 representative protein biomarkers form the B. burg. proteome identified in clinically diagnosed, but STTT seronegative LD patients vs. healthy endemic controls. The data was obtained with the multiplexed magnetic bead-based assay. The antigenic proteins show varying, statistically significant differences in antibody reactivity between the two donor cohorts.

FIG. 7 is a plot showing a receiver operator curve obtained using a general linear model with the Elastic Net regularization. A random 90:10 split was applied to the dataset to train and validate the model over 10-fold cross-validation. The black dots represent a mean ROC, while the curves are the ROC for individual cross-validations. CI-confidence interval.

FIG. 8 are plots showing a comparison of antibody binding profiles to the protein biomarkers indicates minor cross-reactivity between clinically diagnosed, seronegative LD patients and the look-alike diseases used in the study.

### DESCRIPTION

This disclosure is not limited to the particular systems, reaction mixtures, kits, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein the terms “treat”, “treated”, or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

As used herein, “classifier” generally refers to algorithm computer code that receives, as input, data and produces, as output, a classification of the input data as belonging to one or another class.

As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject.

As used herein, “electronic neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets.

As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

As used herein, “reaction mixture” refers a mixture that comprises molecules that can participate in and/or facilitate a given reaction or assay. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction or assay components.

As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology).

As used herein, “value” generally refers to an entry in a dataset that can be anything that characterizes the feature to which the value refers. This includes, without limitation, numbers, words or phrases, symbols (e.g., + or −) or degrees.

As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

As used herein, the term “binding intensity” or “binding affinity”, typically refers to a strength of non-covalent association between or among two or more entities.

As used herein, the term “quasi-random set of peptides” refers to a set of peptide sequences that is selected from truly random sequences (generated by randomly picking amino acids from an amino acid library) such that they meet a set of synthetic constraints and cover the overall set of possible combinatorial sequences as evenly as possible based on criteria such as maximizing the number of the possible n-mers that could be made (n being a number less than the number of residues in the protein, e.g. n=4).

As used herein, the term “in some embodiments” refers to embodiments of all aspects of the disclosure, unless the context clearly indicates otherwise.

As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

As used herein, “protein” or “polypeptide” refers to a polymer of typically more than 50 amino acids attached to one another by a peptide bond. Examples of proteins include enzymes, hormones, antibodies, peptides, and fragments thereof.

As used herein, “peptide” refers to a sequence of 2-50 amino acids attached one to another by a peptide bond. These peptides may or may not be fragments of full proteins. Examples of peptides include KPLEEVLN (SEQ ID NO: 127), FLPFQQK (SEQ ID NO: 128), etc.

As used herein, “system” in the context of analytical instrumentation refers a group of objects and/or devices that form a network for performing a desired objective.

As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

The present disclosure generally describes systems and methods for identifying biomarkers that could be used in the diagnosis and treatment of a disease, such as LD. Biomarkers that may be relevant to disease diagnosis and treatment can include the peptides and/or proteins listed in TABLE 7, TABLE 8, TABLE 9, TABLE 10, and TABLE 11, either individually or in any combination thereof. These peptides may be used to detect the presence of antibodies in a subject infected with B. burgdorferi.

This invention may include other markers that similarly provide information about the underlying immune network and is not restricted to the specific biomarker examples provided herein. The methodology and assay resulting from the discovery of biomarker signatures may be used as the sole evaluation for a subject, or alternatively, may be used in combination with other diagnostics and treatment methodologies.

The biomarkers described herein may be useful for predictive purposes, diagnostic purposes, treatment purposes, for methods for predicting treatment response, methods for monitoring disease progression, and methods for monitoring treatment progress. Further applications of the LD biomarkers include assays as well as kits for use with the methods described herein.

As used herein, a “sample,” such as a biological sample, is a sample obtained from a subject. As used herein, biological samples include all clinical samples including, but not limited to, cells, tissues, and bodily fluids, such as saliva, tears, breath, and blood; derivatives and fractions of blood, such as filtrates, dried blood spots, serum, and plasma; extracted galls; biopsied or surgically removed tissue, including tissues that are, for example, unfixed, frozen, fixed in formalin and/or embedded in paraffin; milk; skin scrapes; nails, skin, hair; surface washings; urine; sputum; bile; bronchoalveolar fluid; pleural fluid, peritoneal fluid; cerebrospinal fluid; prostate fluid; pus; or bone marrow. In a particular example, a sample includes blood obtained from a subject, such as whole blood or serum. In another example, a sample includes cells collected using an oral rinse. Methods for diagnosing, predicting, assessing, and treating CLD in a subject include detecting the presence or absence of antibodies to one or more biomarkers described herein, in a subject's sample. The sample may be isolated from the subject and then directly utilized in a method for determining the presence or absence of antibodies, or alternatively, the sample may be isolated and then stored (e.g., frozen) for a period of time before being subjected to analysis.

Another embodiment of the invention includes an assay and/or kit for diagnosing LD comprising reagents, probes, buffers; antibodies or other agents that enhance the binding of a subject's antibodies to biomarkers; signal generating reagents, including but not limited to fluorescent, enzymatic, electrochemical; or separation enhancing methods, including but not limited to beads, electromagnetic particles, nanoparticles, binding reagents, for the detection of a combination of two or more biomarkers indicative thereof. In some embodiments, the probe and the signal-generating reagent may be one in the same. Techniques of use in all of these methods are discussed below.

### Machine Learning-Based Biomarker Identification

For purposes of assessment and evaluation, choice of biomarkers could be based on evidence of ability to separate subjects that have a disease from controls in a t-test, a receiver operating characteristic (ROC) curve, or that are known to be produced or related to early immune responses. The ROC curve or table is a statistical tool commonly used to evaluate the utility in clinical diagnosis of a proposed assay. The ROC addresses the sensitivity and the specificity of an assay. Therefore, sensitivity and specificity values for a given combination of biomarkers are an indication of the accuracy of the assay. The ROC curve is the most popular graphical tool for evaluating the diagnostic power of a clinical test. Further, a number representing the fraction of the total graphical area under the curve (AUC) can be derived therefrom, which is a widely used method of evaluating a potential diagnostic tool. Sometimes the AUC of a subset of the space is used. This type of evaluation looks at the sensitivity at each specificity of the test. Sensitivity relates to the ability of a test to correctly identify a condition, while specificity relates to the ability of a test to correctly exclude a condition. The present processes and systems described herein can use this type of analysis to identify and evaluate a unique biomarker that may be effectively used in the diagnosis of CLD.

The present disclosure generally describes the use of quasi-random sequence peptide arrays as a tool for comprehensively characterizing the immune response to disease, such as LD. This is based on the realization that a very sparse sampling of the overall sequence dependence of binding of total Immunoglobulin-G (IgG) or Immunoglobulin-M (IgM) in serum allows one, using machine learning methods, to determine a relationship that defines the immune response to all possible sequences. This relationship can then be used to produce a map of which proteins and epitopes in a pathogen, or even in a human in the case of autoimmunity, are responsible for the immune response. Those proteins/epitopes are potential biomarkers for the disease that can be used in a variety of serological assays, such as Luminex.

Although the specific example described herein is in the context of identifying biomarkers for LD and the use of the identified biomarkers for the diagnosis and treatment of LD, one skilled in the art would recognize that the systems and techniques described herein could also be used in the context of other diseases.

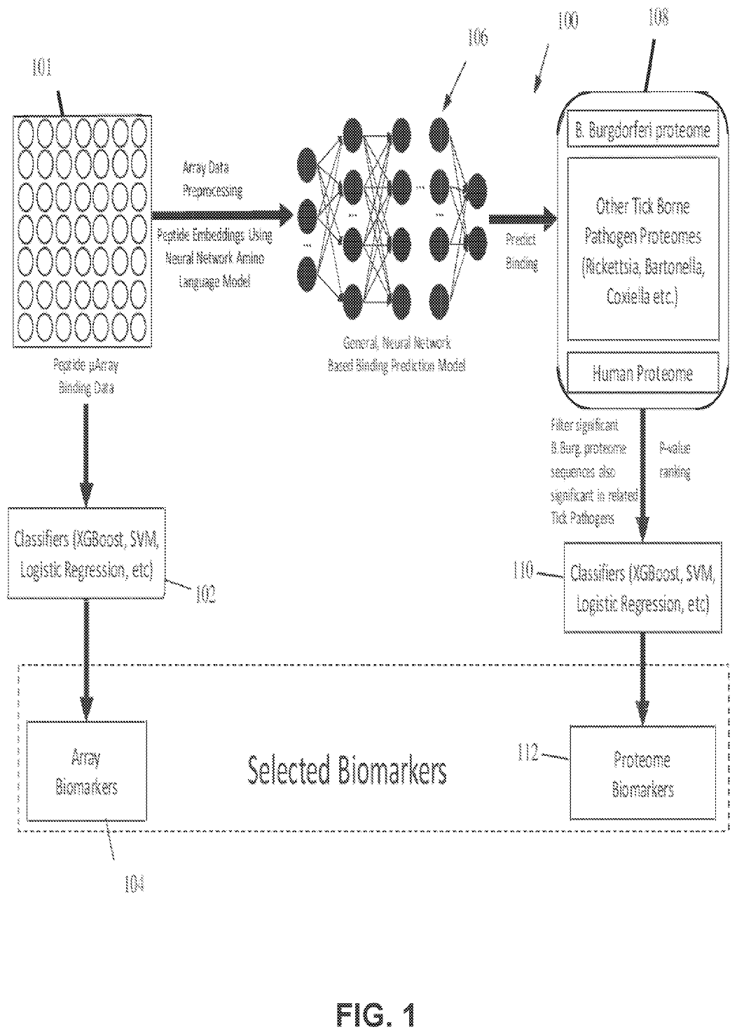

Referring now to FIG. 1, there is shown a diagram of the process 100 described herein for identifying biomarkers for a disease using machine learning techniques. In one embodiment, the process 100 is used for the identification of biomarkers associated with LD, but, as discussed above, this implementation is simply for illustrative purposes and the techniques are not limited solely to LD. The process 100 generally includes: (i) developing one or more classification models 102 (i.e., classifiers) that can distinguish between samples that are from confirmed cases of the diseases, unconfirmed (clinically diagnosed, but seronegative) cases, and healthy controls; and (ii) identifying potential serologic biomarkers that could be used for disease diagnostics using the classification models 102. The diseases can be associated with a vector, such as B. burgdorferi as with LD, and/or a carrier, such as the blacklegged tick as with LD.

In one general embodiment, the process 100 can include obtaining peptide microarray data 101 associated with a microarray including a quasi-random set of peptides. Experimentally, a quasi-random set of 126,000 peptides was used in the examples described below. The microarray data 101 is to be input to a predictive model 106 trained/developed to predict binding intensities associated with the peptide microarray data 101. The peptide microarray data 101 can be obtained using one or more antibodies, such as the anti-IgG secondary antibody. In one embodiment, the process 100 can further include preprocessing the peptide microarray data 101 to place the data in a format suitable for input to the predictive model 106. For example, the peptide microarray data 101 can be processed through a neural network amino language model trained/developed to map the peptide microarray data to a set of embeddings. Various techniques for mapping an input to a set of embeddings are known in the art. The embeddings can then be provided as input to the predictive model 106. The process 100 can further include processing the peptide microarray data 101 (e.g., or embeddings mapped therefrom) through the predictive model 106 to predict the binding intensities with one or more proteomes 108, such as a proteome associated with a vector of the disease (e.g., for LD, the B. burgdorferi proteome), other pathogen proteomes associated with a carrier of the disease (e.g., for LD, other tick-borne pathogen proteomes, such as Rickettsia, Bartonella, or Coxiella bacteria), and/or the human proteome. The process 100 can further include providing the predicted binding intensities output by the predictive model 106 to one or more classifiers 110 that have been trained/developed using one or more potential biomarkers to distinguish between LD cases and negative/healthy controls. The performance of the classifiers 110 can then be assessed to determine whether the particular set of potential biomarkers (e.g., peptides and/or proteins) on which the classifiers 110 were trained performs adequately. If a classifier 110 exhibits adequate classification performance, that can indicate that the one or more potential biomarkers on which the classifier 110 was trained may be candidate proteome biomarkers 112 that could be used to diagnose the disease. In one embodiment, the predicted peptide/protein binding intensities output by the predictive model 106 can be ranked according to their p-values and then selected subsets of the ranked predicted peptide/protein binding intensities can be used to develop/train the classifiers 110. In one embodiment, significant proteome sequences associated with a vector that are also significant in related pathogens (e.g., other pathogens that share the same carrier) can be filtered from the output of the predictive model 106.

A variety of different classification models can be used in the process 100. These classification models can include the one or more classifiers 102 configured to determine the array biomarkers 104 and/or the one or more classifiers 110 configured to determine the proteome biomarkers 112, which are illustrated in FIG. 1, for example. In one embodiment, the classification model can include a general linear model (GLM) with ElasticNet regularization. ElasticNet regularization is a regularized regression method that linearly combines the L1 and L2 penalties of the lasso and ridge methods. Other embodiments can use ridge regression, lasso, and other regularization techniques. In another embodiment, the classification model can include a support vector machine (SVM). In another embodiment, the classification model can include extreme gradient boosting (XGBoost). XGBoost is an open-source software library which provides a gradient boosting framework that functions by generating a prediction model that is an ensemble of weak prediction models (typically, decision trees). In yet another embodiment, the process 100 can include developing multiple classification models in various combinations with each other.

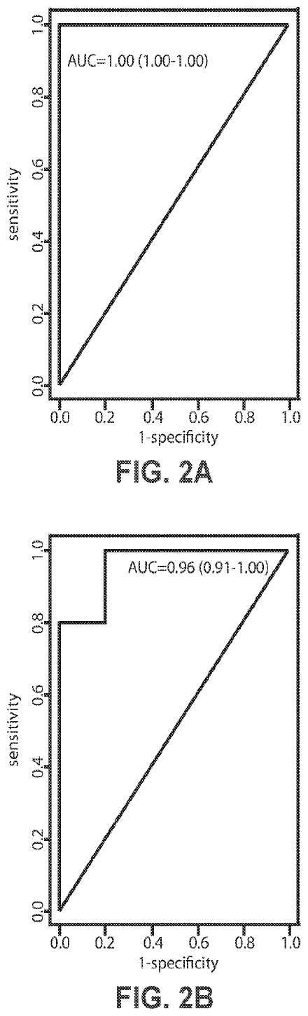

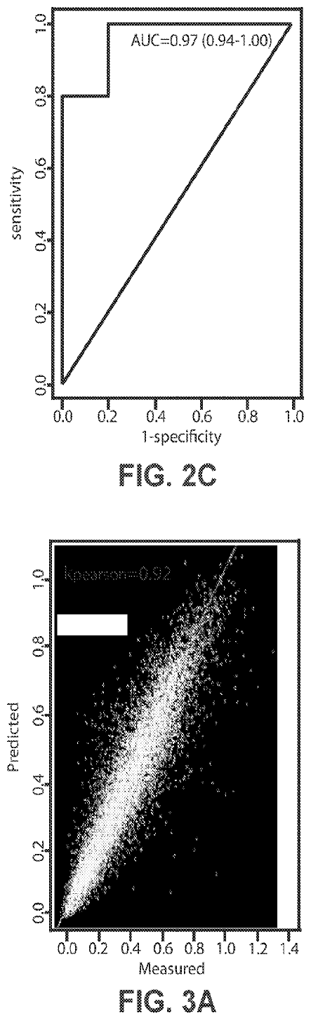

The classifiers can be trained and their performances can be assessed using data from the diverse peptide microarrays obtained using total IgG or IgM present in a subject's serum. FIGS. 2A-2C illustrate ROC curves for GLM-based classifiers trained to distinguish between the confirmed cases and endemic controls using anti-IgG (FIG. 2A) and anti-IgM (FIG. 2B) secondary antibody for measuring antibody binding to the microarray of diverse peptides. Also shown is the ROC for unconfirmed cases vs. endemic controls using an IgG secondary antibody (FIG. 2C). Further, the AUC values along with the 95% confidence intervals are shown. In this particular implementation, the GLM-based classifiers went through 50 training iterations using randomly selected fractions of the dataset with a 90:10 training/validation split. As can be seen, the GLM-based classifiers were robust for anti-IgG and anti-IgM secondary antibodies and results for unconfirmed vs. controls also provided AUC of approximately 0.97 for IgG and IgM. The selected array peptides used to train the classifiers to differentiate between clinically diagnosed, seronegative LD and healthy controls are set forth in TABLE 1 below.

TABLE 1

| SEQ ID NO: | Sequence |
|---|---|
| 1 | KQKGSESE |
| 2 | QKNYYWKED |
| 3 | YHVKPAVVEG |
| 4 | AARGFRED |
| 5 | PPKKVPDG |
| 6 | RLYWGSERSE |
| 7 | AARASEVSE |
| 8 | RFSQAQQSE |
| 9 | AAKGVRDG |
| 10 | KQQASDG |
| 11 | FDVSQFNSD |
| 12 | KDRGEPSE |
| 13 | KLSPQEGAPRSG |
| 14 | KDKAANRFE |
| 15 | DDKHKRFSE |
| 16 | KLKLGQVPEG |
| 17 | HGERAKVFE |
| 18 | KFGVLAEPAE |
| 19 | KYNFEERLG |
| 20 | RQKYDAQEG |
| 21 | QHVNAKAKVE |
| 22 | YRYFWKKSE |
| 23 | DNKDGRGAVE |
| 24 | NYVDHVLNHEG |
| 25 | DHKENVSEG |
| 26 | DNKDKFDPVG |
| 27 | QKKHLFPRPAD |
| 28 | DKKLNSG |
| 29 | KFSLNAKFG |
| 30 | LWNKKPAKD |
| 31 | AARQREVSE |
| 32 | HRREKDG |
| 33 | LYKSGNKYNVD |
| 34 | ELHFYNKDG |
| 35 | RREPNLDYLS |

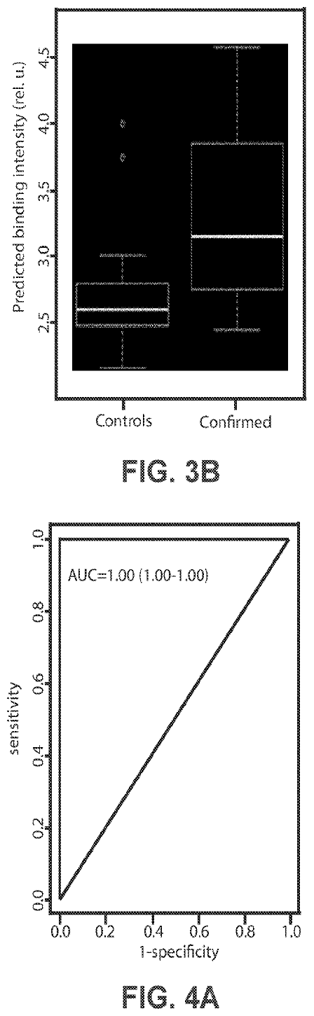

In order to identify potential serologic biomarkers that could be used for Lyme disease diagnostics, the process 100 can further include training a predictive model 106 on the microarray data 101. The predictive model 106 can include one or more deep neural networks, for example. In one embodiment, the deep learning regression model can be trained on the microarray data 101 obtained with the anti-IgG secondary antibody. The model can then be used to predict peptide binding intensities of the entire B. burgdorferi proteome with the goal of identifying potential biomarkers (e.g., peptides and/or proteins) that could be used for Lyme disease detection. In one embodiment, the predictive model 106 can include a set of deep neural networks. In particular, each of the deep neural networks can be developed for each of the samples used in the predictive model 106 development process. The set of deep neural networks can thereafter be used to predict binding of the tiled peptides from the B. burgdorferi proteome. In one embodiment, as a check of model performance, the deep learning model can be trained on a portion of the IgG binding data (i.e., the training data) and can then be validated by being used to predict the portion of the data that was left out of training (i.e., the validation data). As shown in FIG. 3A, the regression model trained on anti-IgG diverse array data to predict measured binding intensities had a strong predictive performance on the validation data, indicated by the Pearson correlation coefficient (k_{Pears}) being 0.92.

The predictions output by the predictive model 106 include canonically surface exposed antigens such as the flagellar motor switch protein along with tRNA ligase proteins for which partially protective antibodies are detected in a murine model of Streptococcus pneumonia. See, e.g., Y. Magez et al., Streptococcus pneumoniae Surface-Exposed Glutamyl tRNA Synthetase, a Putative Adhesin, Is Able to Induce a Partially Protective Immune Response in Mice, The Journal of Infectious Diseases, Volume 196, Issue 6, 15 Sep. 2007, Pages 945-953. In order to validate the predictive model 106, the binding intensities to the V1sE C6 peptide were analyzed to assess the overall ability of the set of deep neural networks to predict known biologically relevant Lyme disease antigens. As shown in FIG. 3B, the trained predictive model 106 accurately predicted strong binding to the C6 peptide GKFAVKDGEK (SEQ ID NO: 126) (p-value=3.49E-9) in the confirmed Lyme cases as compared to the endemic controls. Therefore, the trained predictive model 106 accurately predicts the significantly stronger binding to the C6 peptide in the confirmed cases as opposed to the endemic controls.

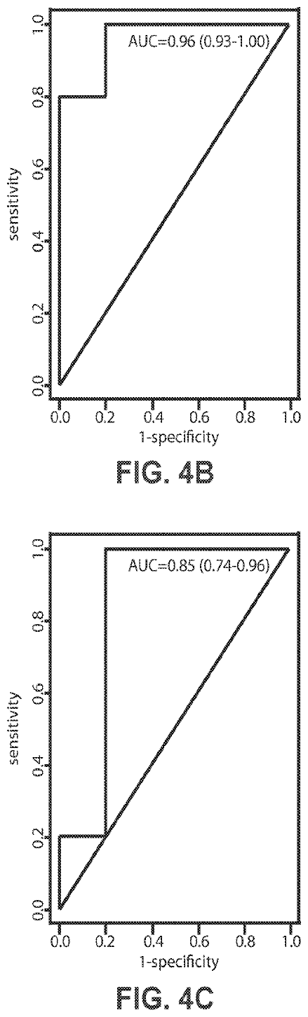

The separate proteome peptides can then be ranked based on the predicted binding intensity distributions between the confirmed Lyme cases and the endemic controls. In one embodiment, the proteome peptides can be ranked based on p-values calculated using Welch's t-test when comparing predicted binding intensity distributions between the confirmed Lyme cases and the endemic controls. Using these calculations, a total of 1,785 peptides with statistically significant mean predicted binding intensities (using a significance level of 0.05 and the Bonferroni correction for multiple comparisons) were identified. Accordingly, the predicted binding intensities of these peptides can be used to develop a classifier model to distinguish the two sample categories. In particular, classifiers can be trained to distinguish between (a) the acute LD cases and controls and (b) clinically diagnosed, seronegative LD cases and controls using predicted bindings of the entire B. burgdorferi proteome. A variety of different classification models can be used. FIG. 4A shows the ROC curve and AUC value for a GLM classifier with ElasticNet regularization developed using the thirty-five highest ranked peptides according to their p-values. FIG. 4B shows the ROC curve and AUC value for a SVM classifier developed using the five highest-ranked peptides according to their p-values. FIG. 4C shows the ROC curve and AUC value for a GLM classifier with ElasticNet regularization developed using the fifteen high-ranked peptides according to their p-values. Based on the various classification models that were implemented and the different numbers of peptides used in conjunction with the classification models, it was determined that the best performance was achieved when using the first five peptides listed in TABLE 2 for a GLM classifier with ElasticNet regularization. Accordingly, it was determined that these five peptides had the strongest predictive power for the patient having acute LD. However, it should be noted that the present disclosure is not limited to embodiments using only these five peptides as biomarkers and the above discussion of the techniques for assessing the predictive power of various combinations of peptides is provided solely for illustrative purposes.

TABLE 2

| SEQ | | | |
|---|---|---|---|
| ID | | UniProt | Protein |
| NO: | Sequence | ID | |
| 36 | IIYRKNEEFI | O51239 | Flagellar motor switch |
| | | | protein |
| 37 | IFNKKDNVVY | P94284 | Thioredoxin reductase |
| 38 | KKFIIDHTKE | O51598 | Uncharacterized protein |
| 39 | IKLIKDIHKD | Q9RZW8 | Adenine specific DNA |
| | | | methyltransferase |
| 40 | KNFIKDVLKD | Q9S0D8 | Uncharacterized protein |
| 41 | LLPFFQKKGYVS | O51345 | Glutamate--tRNA ligase |
| 42 | NKFFDKKDNKEV | O51267 | Leucine--tRNA ligase |
| 43 | AKKYFKENNPES | O51161 | Penicillin-binding protein |
| 44 | KKYNIKFKIDEE | O50742 | Putative phage terminase |

Further, it was determined that different combinations of peptides from the B. burgdorferi proteome can be used to develop different classifiers with similar performance characteristics. For example, TABLE 3 shows a set of selected peptides and corresponding proteins used to develop a SVM classifier to distinguish between the confirmed cases and endemic controls using the B. burgdorferi proteome, as shown in FIG. 4B. As another example, TABLE 4 shows a set of selected peptides and corresponding proteins used to develop a SVM classifier to distinguish between the confirmed cases and endemic controls using the B. burgdorferi proteome. TABLE 5 shows a set of selected peptides and corresponding proteins used to develop a GLM classifier to distinguish between the clinically diagnosed, seronegative LD cases and endemic controls using the B. burgdorferi proteome, as shown in FIG. 4C.

TABLE 3

| SEQ | | | |
|---|---|---|---|
| ID | | UniProt | |
| NO: | Sequence | ID | Protein |
| 40 | KNFIKDVLKD | Q9S0D8 | Uncharacterized protein |
| 37 | IFNKKDNVVY | P94284 | Thioredoxin reductase |
| 45 | KIFFSKEGKE | O51731 | Thymidine kinase |
| 46 | KYFIKPDKMM | O51410 | Lipopolysaccharide |
| | | | biosynt.-relat. protein |
| 47 | KIFLKDEKSV | O51287 | Uncharacterized protein |

TABLE 4

| SEQ | | | |
|---|---|---|---|
| ID | | UniProt | |
| NO: | Sequence | ID | Protein |
| 48 | DFIKKELSKE | O51238 | Alanine--tRNA ligase |
| 45 | KIFFSKEGKE | O51731 | Thymidine kinase |
| 37 | IFNKKDNVVY | P94284 | Thioredoxin reductase |
| 49 | WKKDIENKIS | O51416 | Lipoprotein, putative |
| 50 | KPFFKDLNKE | O51720 | Uncharacterized protein |
| 40 | KNFIKDVLKD | Q9S0D8 | Uncharacterized protein |
| 51 | FFDKKDNKEV | O51267 | Leucine--tRNA ligase |
| 52 | ASKLFKIEEK | O51498 | DNA polymerase I |

TABLE 5

| SEQ | | | |
|---|---|---|---|
| ID | | UniProt | |
| NO: | Sequence | ID | Protein |
| 53 | KTEEELKKKQ | O50870 | Virulent strain-associated |
| | | | repetitive antigen A (VraA |
| | | | protein) |
| 54 | MKIENKDLKF | O51765 | NAGPA domain-containing |
| | | | protein |
| 56 | NKVEDYKKKY | O51173 | Flagellar hook-associated |
| | | | protein 2 |
| 57 | IIKGKVLVEL | O51153 | Rib |
| 58 | KGENIKEFDK | O51161 | Penicillin-binding protein |
| 59 | KKEANFGIPS | O51138 | Replicative DNA helicase |
| 61 | AANEMKDRIK | O51552 | DNA helicase |
| 55 | KIENKDLKFT | O51765 | NAGPA domain-containing |
| | | | protein |
| 62 | TEKNIKVEIY | P70837 | Uncharacterized lipo- |
| | | | protein BBD10 |
| 63 | AGKNIIDPKR | O51568 | Transcription-repair- |
| | | | coupling factor |
| 64 | DKDNIIKILK | O50966 | BBC01 |
| 65 | DKIKEVKVSL | O51486 | Zinc protease, putative |
| 67 | DKIKALFISS | O51099 | Membrane protein, putative |
| 60 | ASISKSLKEL | O51138 | Replicative DNA helicase |
| 68 | MQIKSNLEIK | O50957 | Complement regulator- |
| | | | acquiring surface protein |
| | | | 1 (CRASP-1) |

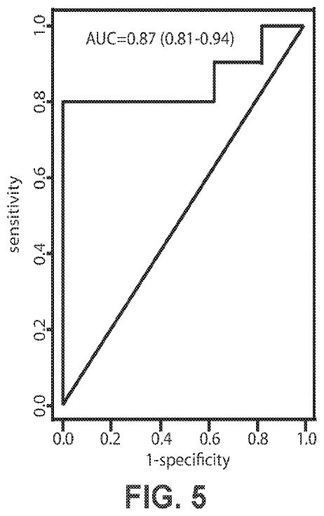

Further, in one embodiment, confirmed and unconfirmed (clinically diagnosed, seronegative) acute LD cases can be combined into a single category and a classifier can be developed/trained to distinguish the combined category from endemic controls. For example, TABLE 6 shows a set of selected peptides and corresponding proteins used to develop a GLM classifier with ElasticNet regularization to distinguish the combined category from endemic controls. Experimentally, the developed GLM classifier resulted in a classification performance of AUC=0.87, as shown in FIG. 5, when utilizing the six highest-ranked peptides by p-values from the B. burgdorferi proteome.

TABLE 6

| SEQ | | | |
|---|---|---|---|
| ID | | UniProt | |
| NO: | Sequence | ID | Protein |
| 40 | KNFIKDVLKD | Q9S0D8 | Uncharacterized protein |
| 69 | IKGAFDGIKN | Q9S093 | Uncharacterized protein |
| 70 | CKEFKKEDFK | O51544 | UDP-N-acetylenol- |
| | | | pyruvoylglucosamine |
| | | | reductase |
| 71 | KDFIKNSEKL | O50692 | Putative lipoprotein |
| 37 | IFNKKDNVVY | P94284 | Thioredoxin reductase |

As can be seen, various combinations of peptides and/or proteins from the B. burgdorferi proteome can be utilized in various combinations to develop various different types of classifiers to identify acute LD cases. Accordingly, various combinations of peptides and/or proteins from the B. burgdorferi proteome can be utilized as diagnostic biomarkers in stand-alone diagnostic tests or in combination with the existing modalities for acute Lyme disease diagnosis. In sum, analysis using the various techniques described above resulted in 26 unique proteins, which are set forth in TABLE 7 from the B. burgdorferi proteome that can be used as biomarkers to diagnose acute LD. Further, 34 peptides, which are set forth in TABLE 8, are selected from the peptide library present on the arrays and can also be used as diagnostic biomarkers for acute LD. In addition, TABLE 9 and TABLE 10 list peptides and proteins from the B. burgdorferi proteome, correspondingly, that were selected for validation using an orthogonal assay based on the Luminex magnetic beads technology. These peptides and proteins can be used as stand-alone biomarkers or in combination with the biomarkers listed in TABLE 7, 8, 9, 10, and/or 11. One skilled in the art would recognize any combination of the listed proteins and/or peptides, including any subset or the entirety of the listed proteins and/or peptides, could be used to develop classifiers or other binary tests for LD detection and diagnosis. Stated differently, any combination of the listed proteins and/or peptides could be used as biomarkers for the detection and diagnosis of LD.

TABLE 7

| Sequence | UniprotID |
|---|---|
| Exported protein A (EppA) | H7C7L7 |
| Flagellar biosynthesis protein FlhA | Q44909 |
| Phage terminase, large subunit, pbsx family | Q9SOD6 |
| Penicillin-binding protein | O51674 |
| Virulent strain-associated repetitive antigen A | O50870 |
| ErpG protein | H7C7N9 |
| S1 antigen | O50899 |
| Basic membrane protein B | Q45011 |
| Complement regulator-acquiring surface protein 1 (CRASP-1) | O50957 |
| Outer membrane protein | O51735 |
| Ribonuclease Y | O51457 |
| Borrelia membrane protein P13 | Q9S011 |
| Flagellar hook protein FlgE | Q44767 |
| Complement regulator-acquiring surface protein 2 (CRASP-2) | O50665 |
| ErpQ protein | Q9S035 |
| Outer membrane protein | O51189 |
| Immunogenic protein P37, putative | O50848 |
| Borrelia outer surface protein E | Q9SOE0 |
| Immunogenic protein P37 | O50846 |
| Outer surface protein D | H7C7Q2 |
| Decorin-binding protein B | POCL68 |
| Outer surface protein B | P17739 |
| Fibronectin-binding protein BBK32 | O50835 |
| Outer membrane efflux protein | O51167 |
| Outer surface protein C | Q07337 |
| PF-32 protcin | Q9S040 |

TABLE 8

| SEQ ID | |
|---|---|
| NO: | Sequence |
| 72 | HSLDQVLQKRG |
| 73 | KQEGAQLPNRE |
| 74 | DKDFANKE |
| 75 | PKKANASD |
| 76 | QEGKQEAG |
| 77 | PWKSFFPQED |
| 78 | PKGGNAKVS |
| 79 | HQEGASGDG |
| 80 | VKKFAQKD |
| 81 | SQLAKSDDG |
| 82 | QRYLRGRLS |
| 83 | FEVFFDLD |
| 84 | LSRSRGYFG |
| 85 | FYFLFAKSD |
| 86 | YSASRLYFSG |
| 87 | YRPQRSLSYFS |
| 88 | QDAAEWKLE |
| 89 | YGQEGGWNQRE |
| 90 | FKFLRLFG |
| 91 | YRALNQFRLF |
| 92 | VGAFALVFG |
| 93 | LQLYLEDFD |
| 94 | FYDYLFEPD |
| 95 | FFLDFED |
| 96 | LFFVDEG |
| 97 | PDYLFDFLFE |
| 98 | AFLLLSE |
| 99 | YFYFDPYPVG |
| 100 | VFFEFDNVLE |
| 101 | RSYAGYRQLS |
| 102 | YRALNQFRLF |
| 103 | FFFEQVED |
| 104 | SYRREDKFEG |
| 105 | FLAFSEPYED |

TABLE 9
(BIOMARKERS-PEPTIDES SELECTED FOR VALIDATION)

| SEQ | | | |
|---|---|---|---|
| ID | | | Uniprot |
| NO: | Sequence | Protein | ID |
| 106 | TKENTITVQQ | Outer surface protein A (OspA) | P0CL66 |
| 36 | IIYRKNEEFI | Flagellar motor switch protein, FliG | O51239 |
| 107 | IKYNKNDLEKEVA | Bacterial extracellular solute-binding protein | O50927 |
| 108 | DNKKGIINGL | Integral outer membrane protein p66 | H7C7N8 |
| 109 | KQWNCNDKKC | Synthetic | In Silico |
| 110 | KIWYMNEKGE | Synthetic | In Silico |
| 111 | KKSFIKELDIEST | Neutrophil activating protein A (NapA) | O51633 |
| 129 | CKNFATGKDIKON | ErpQ protein | Q9S035 |
| 112 | KKLNLMDEKFP | Chemotaxis protein methyltransferase CheR | O51069 |
| 113 | KFAVKDGEKEKAE | Variable large protein VlsE | G5IXI6 |
| 114 | HMACNWKEPL | Synthetic | In Silico |
| 115 | KTTANGEKFDMMA | Probable endolytic peptidoglycan transglycosylase RlpA | O51677 |
| 40 | KNFIKDVLKD | Uncharacterized protein (BB_R43) | Q9S0D8 |
| 116 | TGFKKIRAEFED | Flagellar basal-body rod protein FlgG (Distal rod protein) | O51715 |
| 71 | KDFIKNSEKL | putative lipoprotein (BB_H37) | O50692 |
| 117 | HQTKQFKCVE | Synthetic | In Silico |
| 37 | IFNKKDNVVY | Thioredoxin reductase TrxB | P94284 |
| 118 | KKWALKEGLKETD | LptD Lipopolysaccaride assembly membrane protein | O51778 |
| 119 | KNTYFQEHKD | Synthetic | In Silico |
| 120 | EGVQQEGAQQ | Flagellar filament 41 kDa core protein (Flagellin) FlaB | P11089 |
| 121 | INLDKAQQKL | Borrelia P83/P100 antigen | Q45013 |
| 69 | IKGAFDGIKN | Uncharacterized protein (BB_O15) | Q9S093 |
| 66 | GKFFKKSLDDKDI | Zinc protease, putative | O51486 |
| 70 | CKEFKKEDFK | UDP-N-acetylenolpyruvoylglucosamine reductase MurB | O51544 |
| 122 | NKFFDKKDNKEVTQ | Leucine--tRNA ligase (Leucyl-tRNA synthetase) LeuRS | O51267 |
| 123 | IFTKKLKEDKLDK | DNA polymerase I (PolA) | O51498 |
| 124 | KKYEKKLEEEPSN | Putative surface protein, Involved in colonization BBE31 | O50725 |
| 125 | HKMMILGREDD | Putative phage portal protein, HI1409 family | O50743 |

TABLE 10
(BIOMARKERS-PROTEINS SELECTED FOR VALIDATION)

| Protein | UniprotID |
|---|---|
| UDP-N-acetylenolpyruvoylglucosamine reductase MurB | O51544 |
| Flagellar motor switch protein, FliG | O51239 |
| Leucine—IRNA ligase (Leucyl-IRNA synthetase) LeuRS | O51267 |
| Probable endolytic peptidoglycan transglycosylase RlpA | O51677 |
| Thioredoxin reductase TrxB | P94284 |
| Uncharacterized protein (BB_R43) | Q9SOD8 |
| Putative phage portal protein, HI1409 family | O50743 |
| Flagellar basal-body rod protein FlgG (Distal rod protein) | O51715 |
| Borrelia P83/P100 antigen | Q45013 |
| Outer surface protein A (OspA) | POCL66 |
| Bacterial extracellular solute-binding protein, family 5; | O50927 |
| OppAIV | |
| Flagellar filament 41 kDa core protein (Flagellin) FlaB | P11089 |
| Chemotaxis protein methyltransferase CheR | O51069 |
| DNA polymerase I (PolA) | O51498 |
| Integral outer membrane protein p66 | H7C7N8 |
| LptD Lipopolysaccaride assembly membrane protein | O51778 |

In sum, the process 100 described herein includes building one or more classifiers using peptide array data to distinguish between confirmed cases of the disease and negative/healthy controls. Further, the process 100 includes building one or more predictive models to predict binding to a proteome, such as the B. burgdorferi proteome for Lyme disease. Accordingly, the process 100 can be used to identify a set of biomarkers for diagnosing the disease.

### Exemplary Candidate Panel Validation

Our efforts were focused on the validation of a number of candidate biomarkers that were discovered in silico in our preliminary studies, as described herein. The validation was performed using the Luminex magnetic bead-based assays in extended cohorts of clinically diagnosed, symptomatic patients presenting with the erythema migrans (EM) rash of >5 cm in diameter, but seronegative by the CDC-recommended standard two-tier test (STTT), and endemic healthy controls. The main goal of this example was to validate differentiating power of a panel of in silico identified candidate peptide and protein biomarkers to distinguish between the two donor cohorts. In addition, cross-reactivity of the biomarkers was evaluated by measuring their reactivity in a group of patients diagnosed with diseases that show symptoms similar to LD. The choice of clinically diagnosed but seronegative LD patients was made based on the inability of the STTT to detect LD in this cohort of patients. Most of these patients presented with relatively mild symptoms and were believed to be in the early stages of LD. However, it is possible that some of the patients had been infected with other pathogens, such as STARI due to its recent marked spread in the US Northeast endemic areas. We note that all patients in this cohorts tested negative with the STTT and thus would have been left undiagnosed or misdiagnosed by a physician without proper training or awareness of a possible LD diagnosis.

We validated a panel of 30 candidate protein biomarkers from the B. burg. proteome identified previously in our proof-of-concept studies, as described herein. We identified 4 protein biomarkers and demonstrated their differentiating power in distinguishing clinically diagnosed LD patients that were previously missed by the STTT from healthy endemic controls.

Approach for multiplexed analysis of the presence of antibodies against B. burg. To perform validation of the candidate biomarker panel, we coupled the biomarkers to the carboxylated magnetic microbeads using standard bead functionalization protocols.

We focused on the validation of the candidate peptide and protein biomarkers identified in silico in our preliminary studies, as described herein. Here, we used cohorts of donors (N=100 samples per cohort) in the clinically diagnosed (seronegative by STTT) LD and healthy endemic control categories. Our validation was based on the widely used bead-based approach and protocols developed and commercialized by Luminex (Austin, TX). All validation assays were performed on a MagPix instrument. To minimize risks, we used standard bead preparation and functionalization and assay protocols suggested by the vendor.

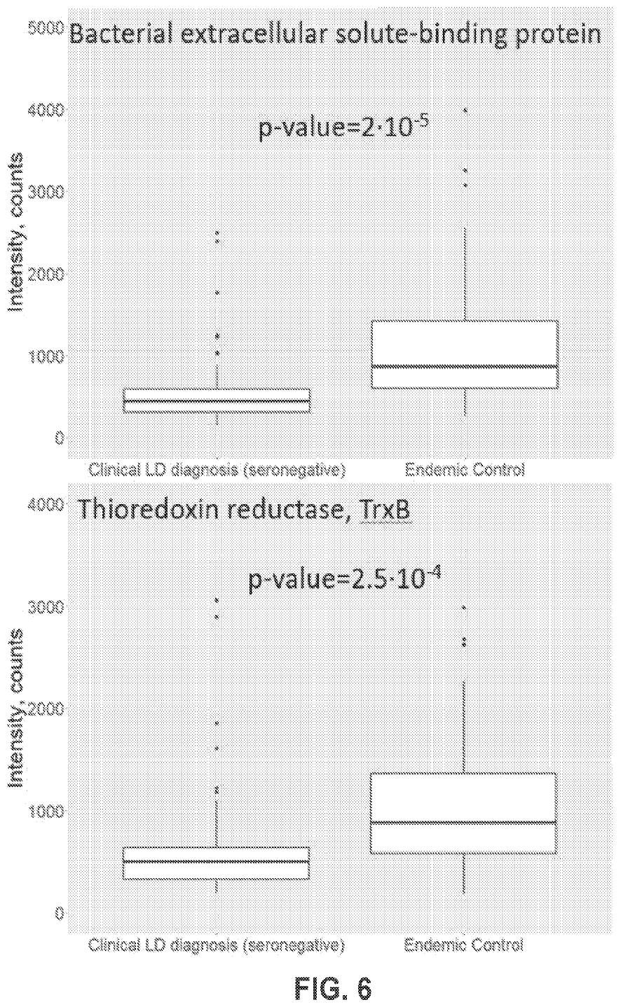

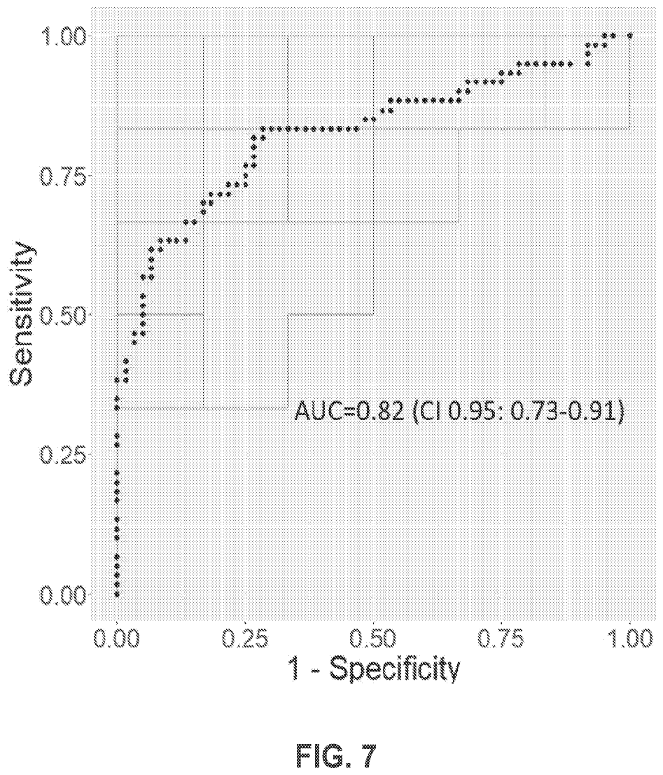

We have examined a panel of candidate biomarkers containing a total of 30 proteins from the B. burg. proteome. The proteins were screened for differentiating performance between the clinically diagnosed, but serologically negative LD, and the endemic healthy controls using the assay protocol outline above. The obtained results revealed a panel of 4 biomarkers with differentiating power between the two cohorts (TABLE 11). Interestingly, all 4 biomarkers exhibited an overall reduced binding in the clinically diagnosed, but STTT-negative, LD as compared to the endemic healthy controls (FIG. 6). Using the obtained data, we have developed a simple classifier based on the general linear model using the Elastic Net normalization with a receiver operating curve (ROC) as shown in FIG. 7. We find classifier can differentiate between the clinically diagnosed, STTT-negative LD and endemic healthy controls with an area under the curve (AUC) of 0.82 (CI 0.95:0.73-0.91) and a resulting sensitivity of 64% at 87.5% specificity. As outlined above, the clinically diagnosed LD cohort may contain patients infected with other pathogens that also present with symptoms similar to LD. Therefore, the actual classification performance could be higher, but needs additional evaluation, ideally using longitudinal samples of patients that seroconvert at a later time point. We note that the 4 biomarkers do not show significant differentiation power between STTT-positive LD patients and endemic controls, suggesting that they are specific to the early stage of LD.

TABLE 11
(VALIDATED BIOMARKERS-PROTEINS)

| SEQ | | | |
|---|---|---|---|
| ID | | | Uniprot |
| NO: | Sequence | Protein | ID |
| 36 | IIYRKNEEFI | Flagellar motor switch | O51239 |
| | | protein, FliG | |
| 115 | KTTANGEKFD | Probable endolytic | O51677 |
| | MMA | peptidoglycan | |
| | | transglycosylase RlpA | |
| 37 | IFNKKDNVVY | Thioredoxin reductase | P94284 |
| | | TrxB | |
| 107 | IKYNKNDLEK | Bacterial extracellular | O50927 |
| | EVA | solute-binding protein, | |
| | | family 5; OppAIV | |

The observed performance is significant in that it correctly identifies 64% with 87.5% specificity of patients that were previously missed by the STTT. Based on this exemplary finding a commercial serologic test based on the newly validated biomarkers would add substantial value to early LD diagnosis.

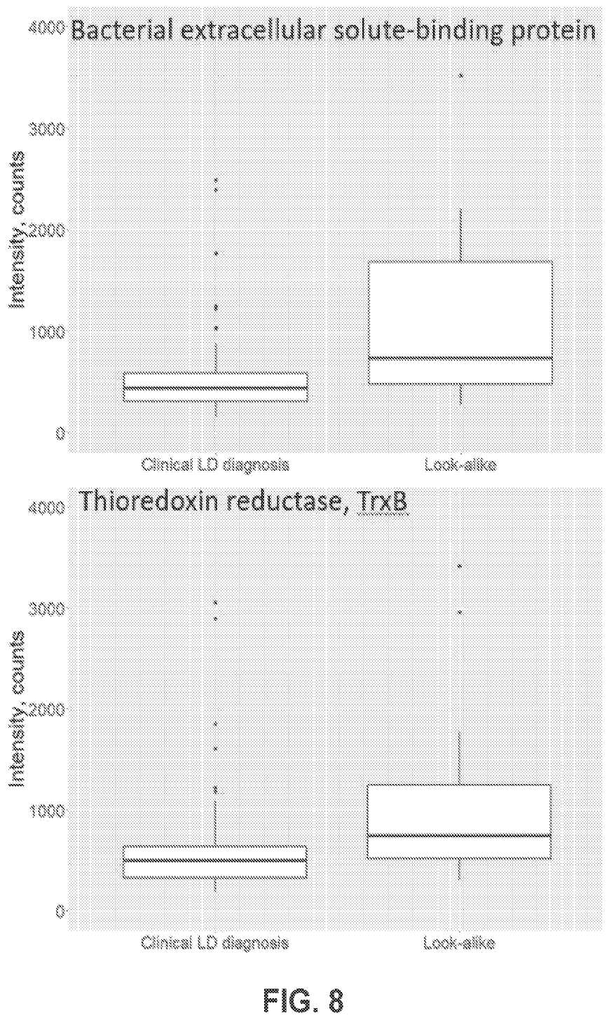

Further, potential cross-reactivity of an optimized biomarker panel was evaluated. To this end, we assayed a total of 15 samples from patients diagnosed with influenza, Babesiosis, rheumatoid arthritis, syphilis, multiple sclerosis, mononucleosis, and severe periodontitis. We find a similar overall trend of binding intensities observed compared with the clinically diagnosed LD with the endemic control samples. Namely, the binding patterns of all 4 validated biomarkers suggest lower antibody reactivity of the clinically diagnosed LD patients than in the look-alike cohort (FIG. 8). The seemingly suppressed antibody reactivity to these targets suggests a potential role of the known immunomodulating activity of B. burg. in the early stages of the disease. Furthermore, we observed two more potential biomarkers that show differentiating power between the two cohorts—integral outer membrane protein p66 (uniprotID: H7C7N8) and Borrelia P83/P100 antigen (uniprotID: Q45013 (SEQ ID NO: 121)). These proteins could be included in a diagnostic test for improved differentiation between clinically diagnosed LD and look-alike diseases. This data suggests that there is only minor cross-reactivity with the look-alike diseases included in this study. However, given the fact that the clinically diagnosed LD cohort contains only patients presenting with an EM>5 cm, which is not typical to the look-alike diseases used in this study, it is expected that the actual differentiation capability of the biomarkers between LD and look-alike diseases is higher. The main result of this example is that the selected biomarkers provide marked differentiation between the clinically diagnosed LD and look-alike diseases used in the study. Two more biomarkers could be added to the assay to improve differentiation performance.

In summary, this example provided valuable data confirming the validity of our approach based on broad and agnostic profiling of the patient's circulating antibody repertoire. In this example, we were able to validate a panel of 4 protein biomarkers with robust power to differentiate between clinically diagnosed LD that were missed by the current STTT and endemic controls.

### Lyme Disease Diagnosis & Treatment

Once the peptides and/or proteins that correspond to the disease have been identified as biomarkers using the techniques describes above, antibodies that bind to these biomarkers can be detected in a patient sample. Accordingly, a treatment decision can be made based on the presence of the antibodies.

In some embodiments, a method for diagnosing a B. burgdorferi infection in a subject in need thereof comprises obtaining a sample from the subject and detecting the presence of antibodies in the subject sample that binds to one or more of the B. burgdorferi antigenic peptides listed in TABLE 7, 8, 9, 10, and/or 11.

In some embodiments, a method of treating a subject with a B. burgdorferi infection comprises obtaining a sample from the subject, detecting the presence of antibodies in the subject sample that binds to one or more of the B. burgdorferi antigenic peptides listed in TABLE 7, 8, 9, 10, and/or 11, and administering an antibiotic composition.

In other embodiments, a method of treating a subject with LD comprises obtaining a sample from the subject, detecting the presence of antibodies in the subject sample that binds to one or more of the B. burgdorferi antigenic peptides listed in TABLE 7, 8, 9, 10, and/or 11, and administering an antibiotic composition.

In some embodiments, the methods disclosed herein are not limited to an infection or a disease caused by B. burgdorferi, but also encompasses diseases caused by other Borrelia species, such as Borrelia burgdorferi sensu stricto, Borrelia azfelii, Borrelia garinii, Borrelia valaisiana, Borrelia spielmanii, Borrelia bissettii, Borrelia lusitaniae, and Borrelia bavariensis.

In some embodiments, subject sample includes all clinical samples including, but not limited to, cells, tissues, and bodily fluids, such as: saliva, tears, breath, blood; derivatives and fractions of blood, such as filtrates, dried blood spots, serum, and plasma. In some embodiments, a suitable subject sample may comprise, for instance, a whole blood sample, or a cerebrospinal fluid sample, or a synovial fluid sample, any of which may be obtained from a subject.

The subject sample may be obtained or isolated by any technique known in the art. While cell extracts can be prepared using standard techniques in the art, the methods generally use serum, blood filtrates, blood spots, plasma, saliva, tears, or urine prepared with simple methods such as centrifugation and filtration. The use of specialized blood collection tubes, such as rapid serum tubes containing a clotting enhancer to speed the collection of serum and agents to prevent alteration of the antibodies is one preferred method of preparation. Another preferred method utilizes tubes containing factors to limit platelet activation, one such tube contains citrate as the anticoagulant and a mixture of theophylline, adenosine, and dipyrimadole.

In some embodiments, detecting the presence of antibodies in the subject sample that binds to one or more of the B. burgdorferi antigenic peptides comprises using any of the immunoassays known in the art, such as ELISA, western blotting, surface plasmon resonance, microarray, and the like. In some embodiments, the immunoassay may be an ELISA. ELISAS are generally well known in the art. In a typical “indirect” ELISA, an antigen having specificity for the antibodies under test is immobilized on a solid surface (e.g. the wells of a standard microtiter assay plate, or the surface of a microbead or a microarray) and a sample comprising bodily fluid to be tested for the presence of antibodies is brought into contact with the immobilized antigen. Any antibodies of the desired specificity present in the sample will bind to the immobilized antigen. The bound antibody/antigen complexes may then be detected using any suitable method. In one embodiment, a labelled secondary anti-human immunoglobulin antibody, which specifically recognizes an epitope common to one or more classes of human immunoglobulins, is used to detect the antibody/antigen complexes. Typically the secondary antibody will be anti-IgG or anti-IgM. The secondary antibody is usually labelled with a detectable marker, typically an enzyme marker such as, for example, peroxidase or alkaline phosphatase, allowing quantitative detection by the addition of a substrate for the enzyme which generates a detectable product, for example a coloured, chemiluminescent or fluorescent product. Other types of detectable labels known in the art may be used.

In the methods disclosed herein, one or more of the B. burgdorferi antigenic peptides listed in TABLE 7, 8, 9, 10, and/or 11 may be immobilized on a solid surface, and a sample from a subject is brought into contact with the immobilized antigen(s). The methods disclosed herein can be used to detect two or more antibodies in a subject's sample comprising a bodily fluid. In some embodiments, the B. burgdorferi antigenic peptides are selected from IIYRKNEEFI (SEQ ID NO: 36), IFNKKDNVVY (SEQ ID NO: 37), KKFIIDHTKE (SEQ ID NO: 38), IKLIKDIHKD (SEQ ID NO: 39), or KNFIKDVLKD (SEQ ID NO: 40).

The methods disclosed herein may be used in predicting and/or monitoring response of an individual to any Lyme disease treatments. In some embodiments, the immunoassays disclosed herein can be used in parallel with other methods of diagnosing Lyme disease, including subjective (e.g., self-report of symptoms) and objective measurements of Lyme disease symptoms. For example, the methods provided herein can be used in parallel with clinical observations of, or a subject's self-reporting of, tick bite, erythema migrans (or bull-eye shaped rash), skin lesion, pain, fever, headache, swelling, or other symptoms associated with Lyme disease.

In some embodiments, the method comprises administering a therapeutic amount of an antibiotic composition. Non-limiting examples of antibiotics that may be administered include tetracyclines, such as oxytetracycline, doxycycline, or minocycline; penicillins, such as amoxicillin or penicillin; cephalosporins, such as cefaclor, cefbuperazone, cefminox, cefotaxime, cefotetan, cefmetazole, cefoxitin, cefuroxime axetil, cefuroxime acetyl, ceftin, or ceftriaxone; macrolides, such as azithromycin, clarithromycin, or erythromycin.

In certain embodiments, the therapeutically effective amount of the antibiotic composition will be from about 500 mg to about 5000 mg daily, about 500 mg to about 4000 mg daily, about 500 mg to about 3000 mg daily, about 500 mg to about 2000 mg daily, about 500 mg to about 1500 mg daily, or about 500 mg to about 1000 mg daily.

In some embodiments, the antibiotic compositions disclosed herein may be administered once, as needed, once daily, twice daily, three times a day, once a week, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks. It will be understood that the specific dose level and frequency of dosage for any particular subject can be varied and will depend upon a variety of factors including the species, age, body weight, general health, gender and diet of the subject, the mode and time of administration, rate of excretion, drug combination, and severity of the particular condition.

Administration can be by any route including parenteral and transmucosal (e.g., oral, nasal, buccal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

Also provided herein are kits including one or more of the compositions provided herein. Instructions for use can include instructions for diagnostic applications of the compositions for diagnosing Lyme disease and/or monitoring the response of a subject to treatment of Lyme disease. The kit can include one or more other elements including: instructions for use and other reagents such as serum-free medium, microtiter plates coated with one or more one B. burgdorferi antigenic peptides listed in TABLE 7, 8, 9, 10, and/or 11, labelled secondary antibodies, a substrate, buffers, and antibiotic compositions. The secondary antibody can be any detectably labeled antibody, for example, an antibody tagged with a fluorescent dye, e.g., an Alexa Fluor 488-conjugated antibody; an enzyme-conjugated antibody, e.g., alkaline phosphatase-conjugated antibody; or an antibody conjugated with one member of a specific binding pair, e.g., an antibody conjugated with biotin or streptavidin. For example, when a biotinylated antibody is included in the kit, the kit also includes enzyme-conjugated streptavidin, e.g., alkaline phosphatase-conjugated streptavidin. The kit can include a chromogenic, fluorogenic, or electrochemiluminescent substrate of the enzyme on the secondary antibody or strepavidin. For example, a chromogenic substrate for alkaline phosphatase can be a 5-Bromo-4-chloro-3-indolyl phosphate (BCIP), nitro blue tetrazolium chloride (NBT), or a mixture of BCIP and NBT. The instructions for use can be in a paper format or on a CD or DVD.

Some further aspects are defined in the following clauses:

Clause 1: A method of detecting Lyme disease in a subject, the method comprising detecting a presence of one or more of the B. burgdorferi antigenic peptides or proteins listed in TABLE 7, 8, 9, 10, and/or 11, and/or a presence of one or more amino acids that encode one or more of the B. burgdorferi antigenic peptides or proteins listed in TABLE 7. 8, 9, 10, and/or 11, in a sample obtained from the subject, thereby detecting Lyme disease in the subject.

Clause 2: The method of Clause 1, wherein detecting the presence of the one or more of the B. burgdorferi antigenic peptides or proteins listed in TABLE 7, 8, 9, 10, and/or 11 comprises detecting a presence of one or more antibodies in the sample that bind to the one or more B. burgdorferi antigenic peptides or proteins listed in TABLE 7, 8, 9, 10, and/or 11.

Clause 3: The method of Clause 1 or Clause 2, wherein detecting the presence of the one or more of the B. burgdorferi antigenic peptides or proteins listed in TABLE 7, 8, 9, 10, and/or 11 comprises the use of antibodies raised against the one or more of the B. burgdorferi antigenic peptides or proteins listed in TABLE 7, 8, 9, 10, and/or 11 in the sample.

Clause 4: The method of any one of the preceding Clauses 1-3, wherein detecting the presence of the one or more amino acids that encode the one or more B. burgdorferi antigenic peptides or proteins listed in TABLE 7, 8, 9, 10, and/or 11 comprises sequencing the one or more nucleic acids that encode the antigenic peptides or proteins in the sample.

Clause 5: The method of any one of the preceding Clauses 1-4, further comprising obtaining the sample from the subject.

Clause 6: The method of any one of the preceding Clauses 1-5, further comprising administering at least one therapeutic treatment to the subject.

Clause 7: The method of any one of the preceding Clauses 1-6, wherein administering the at least one therapeutic treatment comprises administering an effective amount of an antibiotic selected from oxytetracycline, doxycycline, minocycline, amoxicillin, penicillin, cefaclor, cefbuperazone, cefminox, cefotaxime, cefotetan, cefmetazole, cefoxitin, cefuroxime axetil, cefuroxime acetyl, ceftin, ceftriaxone, azithromycin, clarithromycin, erythromycin, and combination thereof.

Clause 8: A reaction mixture comprising reagents for performing the method of any one of the preceding Clauses 1-7.

Clause 9: A kit comprising reagents for performing the method of any one of the preceding Clauses 1-8.

Clause 10: A computer-implemented method of generating predicted binding intensities from a microarray peptide data set, the method comprising: passing the microarray peptide data set through an electronic neural network model, wherein the microarray peptide data set is obtained from a microarray that comprises a quasi-random set of peptides using one or more antibodies or donor serum sample and wherein the electronic neural network model has been trained to predict binding intensities of peptides not present on the microarray; and, outputting from the electronic neural network the predicted binding intensities of peptides not represented on the microarray using the microarray peptide data set.

Clause 11: The computer-implemented method of Clause 10, wherein the electronic neural network model is trained on binding intensities associated with the microarray peptide data set is utilized to predict binding intensities of donor's circulating antibodies to one or more proteomes selected from the group consisting of: a proteome associated with a vector of a disease, a proteome associated with a carrier of a vector of a disease, and a human proteome.

Clause 12: The computer-implemented method of Clause 10 or Clause 11, wherein predicted strong binding targets in the proteome(s) are used to identify immunogenic full proteins that can further be used as biomarkers in orthogonal assays.

Clause 13: The computer-implemented method of any one of the preceding Clauses 10-12, further comprising: passing the predicted binding intensities of peptides that are not present on the microarray set to one or more classifiers that have been trained using one or more potential biomarkers to distinguish between a disease state and a non-disease state.

Clause 14: The computer-implemented method of any one of the preceding Clauses 10-13, further comprising: ranking at least a subset of a set of peptides not represented on the array based upon the predicted binding intensities obtained using a machine learning model trained on the microarray peptide data set to produce a set of ranked peptides; using statistical methods, identifying protein biomarkers from proteomes of the pathogen and/or other associated organisms; producing a classification model using predicted intensity values of the set of ranked peptides that are not represented on the microarray, which classification model classifies a sample from a test subject as being positive or negative for a disease; assessing a performance of the classification model to produce a classification model performance assessment measure; and, determining whether the set of ranked peptides comprises candidate biomarkers for detecting a presence of the disease in test subjects based on the classification model performance assessment measure.

Clause 15: The computer-implemented method of any one of the preceding Clauses 10-14, wherein the disease is Lyme disease.

Clause 16: The computer-implemented method of any one of the preceding Clauses 10-15, wherein the classification model is selected from the group consisting of: a general linear model, a support vector machine, an extreme gradient boosting model, an electronic neural network model, and a combination thereof.

Clause 17: The computer-implemented method of any one of the preceding Clauses 10-16, wherein the disease is associated with a pathogen and a carrier and wherein the method further comprises: filtering, from the subset of the quasi-random set of peptides, carrier-related peptides, wherein the carrier-related peptides are associated with other pathogens associated with the carrier.

Clause 18: The computer-implemented method of any one of the preceding Clauses 10-17, wherein the pathogen is Borrelia burgdorferi and the carrier is the blacklegged tick Ixodes scapularis.

Clause 19: The computer-implemented method of any one of the preceding Clauses 10-18, wherein the subset of the set of peptides not represented on the microarray is ranked according to p-values associated with corresponding predicted binding intensities.

Clause 20: The computer-implemented method of any one of the preceding Clauses 10-19, wherein the subset of peptides not represented on the microarray corresponds to a set of n-highest ranked peptides, wherein n is an integer greater than one.

Clause 21: The computer-implemented method of any one of the preceding Clauses 10-20, wherein assessing the performance of the classification model comprises generating a ROC curve corresponding to the performance of the classification model.

Clause 22: A system for generating predicted binding intensities from a microarray peptide data set using an electronic neural network, the system comprising: a processor; and a memory communicatively coupled to the processor, the memory storing instructions which, when executed on the processor, perform operations comprising: passing the microarray peptide data set through the electronic neural network, wherein the microarray peptide data set is obtained from a microarray that comprises a quasi-random set of peptides using one or more antibodies or donor serum sample and wherein the electronic neural network has been trained to predict binding intensities of peptides not present on the microarray using the microarray peptide data set; and, outputting from the electronic neural network the predicted binding intensities of the peptides not represented on the microarray.

Clause 23: The system of Clause 22, wherein the instructions which, when executed on the processor, further perform operations comprising: passing the predicted binding intensities of the peptides not represented on the microarray to one or more classifiers that have been trained using one or more potential biomarkers to distinguish between a disease state and a non-disease state.

Clause 24: The system of Clause 22 or Clause 23, wherein the instructions which, when executed on the processor, further perform operations comprising: mapping, using an electronic neural network amino language model, the microarray peptide data set to a set of embeddings; and passing the set of embeddings to a machine learning model to determine the predicted binding intensities of peptides not represented on the array.

Clause 25: The system of any one of the preceding Clauses 22-24, wherein the instructions which, when executed on the processor, further perform operations comprising: ranking at least a subset of a set of peptides not represented on the microarray based upon the predicted binding intensities from the microarray peptide data set to produce a set of ranked peptides; producing a classification model using the set of ranked peptides, which classification model classifies a sample from a test subject as being positive or negative for a disease; assessing a performance of the classification model to produce a classification model performance assessment measure; and, determining whether the set of ranked peptides comprises candidate biomarkers for detecting a presence of the disease in test subjects based on the classification model performance assessment measure.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of detecting Lyme disease in a subject, the method comprising detecting a presence of one or more of the B. burgdorferi antigenic peptides or proteins listed in TABLE 7, 8, 9, 10, and/or 11, and/or a presence of one or more amino acids that encode one or more of the B. burgdorferi antigenic peptides or proteins listed in TABLE 7, 8, 9, 10, and/or 11, in a sample obtained from the subject, thereby detecting Lyme disease in the subject.

2. The method of claim 1, wherein detecting the presence of the one or more of the B. burgdorferi antigenic peptides or proteins listed in TABLE 7, 8, 9, 10, and/or 11 comprises detecting a presence of one or more antibodies in the sample that bind to the one or more B. burgdorferi antigenic peptides or proteins listed in TABLE 7, 8, 9, 10, and/or 11; or wherein detecting the presence of the one or more of the B. burgdorferi antigenic peptides or proteins listed in TABLE 7, 8, 9, 10, and/or 11 comprises the use of antibodies raised against the one or more of the B. burgdorferi antigenic peptides or proteins listed in TABLE 7, 8, 9, 10, and/or 11 in the sample; or wherein detecting the presence of the one or more amino acids that encode the one or more B. burgdorferi antigenic peptides or proteins listed in TABLE 7, 8, 9, 10, and/or 11 comprises sequencing the one or more nucleic acids that encode the antigenic peptides or proteins in the sample.

3. (canceled)

4. (canceled)

5. The method of claim 1, further comprising obtaining the sample from the subject.

6. The method of claim 1, further comprising administering at least one therapeutic treatment to the subject.

7. The method of claim 6, wherein administering the at least one therapeutic treatment comprises administering an effective amount of an antibiotic selected from oxytetracycline, doxycycline, minocycline, amoxicillin, penicillin, cefaclor, cefbuperazone, cefminox, cefotaxime, cefotetan, cefmetazole, cefoxitin, cefuroxime axetil, cefuroxime acetyl, ceftin, ceftriaxone, azithromycin, clarithromycin, erythromycin, and combination thereof.

8. A reaction mixture comprising reagents for performing the method of claim 1.

9. A kit comprising reagents for performing the method of claim 1.

10. A computer-implemented method of generating predicted binding intensities from a microarray peptide data set, the method comprising:
passing the microarray peptide data set through an electronic neural network model, wherein the microarray peptide data set is obtained from a microarray that comprises a quasi-random set of peptides using one or more antibodies or donor serum sample and wherein the electronic neural network model has been trained to predict binding intensities of peptides not present on the microarray; and,
outputting from the electronic neural network the predicted binding intensities of peptides not represented on the microarray using the microarray peptide data set.

11. The computer-implemented method of claim 10, wherein the electronic neural network model is trained on binding intensities associated with the microarray peptide data set is utilized to predict binding intensities of donor's circulating antibodies to one or more proteomes selected from the group consisting of: a proteome associated with a vector of a disease, a proteome associated with a carrier of a vector of a disease, and a human proteome.

12. The computer-implemented method of claim 11, wherein predicted strong binding targets in the proteome(s) are used to identify immunogenic full proteins that can further be used as biomarkers in orthogonal assays.

13. The computer-implemented method claim 10, further comprising:
passing the predicted binding intensities of peptides that are not present on the microarray set to one or more classifiers that have been trained using one or more potential biomarkers to distinguish between a disease state and a non-disease state.

14. The computer-implemented method of claim 10, further comprising:
ranking at least a subset of a set of peptides not represented on the array based upon the predicted binding intensities obtained using a machine learning model trained on the microarray peptide data set to produce a set of ranked peptides;
using statistical methods, identifying protein biomarkers from proteomes of the pathogen and/or other associated organisms;
producing a classification model using predicted intensity values of the set of ranked peptides that are not represented on the microarray, which classification model classifies a sample from a test subject as being positive or negative for a disease;
assessing a performance of the classification model to produce a classification model performance assessment measure; and,
determining whether the set of ranked peptides comprises candidate biomarkers for detecting a presence of the disease in test subjects based on the classification model performance assessment measure.

15. The computer-implemented method of claim 14, wherein the disease is Lyme disease.

16. The computer-implemented method of claim 14, wherein the classification model is selected from the group consisting of: a general linear model, a support vector machine, an extreme gradient boosting model, an electronic neural network model, and a combination thereof.

17. The computer-implemented method of claim 14, wherein the disease is associated with a pathogen and a carrier and wherein the method further comprises:
filtering, from the subset of the quasi-random set of peptides, carrier-related peptides, wherein the carrier-related peptides are associated with other pathogens associated with the carrier.

18. The computer-implemented method of claim 17, wherein the pathogen is Borrelia burgdorferi and the carrier is the blacklegged tick Ixodes scapularis.

19. The computer-implemented method of claim 14, wherein the subset of the set of peptides not represented on the microarray is ranked according to p-values associated with corresponding predicted binding intensities; or wherein the subset of peptides not represented on the microarray corresponds to a set of n-highest ranked peptides, wherein n is an integer greater than one.

20. (canceled)

21. The computer-implemented method of claim 14, wherein assessing the performance of the classification model comprises generating a ROC curve corresponding to the performance of the classification model.

22. A system for generating predicted binding intensities from a microarray peptide data set using an electronic neural network, the system comprising:
a processor; and
a memory communicatively coupled to the processor, the memory storing instructions which, when executed on the processor, perform operations comprising:
passing the microarray peptide data set through the electronic neural network, wherein the microarray peptide data set is obtained from a microarray that comprises a quasi-random set of peptides using one or more antibodies or donor serum sample and wherein the electronic neural network has been trained to predict binding intensities of peptides not present on the microarray using the microarray peptide data set; and,
outputting from the electronic neural network the predicted binding intensities of the peptides not represented on the microarray.

23. The system of claim 22, wherein the instructions which, when executed on the processor, further perform operations comprising:
passing the predicted binding intensities of the peptides not represented on the microarray to one or more classifiers that have been trained using one or more potential biomarkers to distinguish between a disease state and a non-disease state; or
mapping, using an electronic neural network amino language model, the microarray peptide data set to a set of embeddings; and
passing the set of embeddings to a machine learning model to determine the predicted binding intensities of peptides not represented on the array; or
ranking at least a subset of a set of peptides not represented on the microarray based upon the predicted binding intensities from the microarray peptide data set to produce a set of ranked peptides;
producing a classification model using the set of ranked peptides, which classification model classifies a sample from a test subject as being positive or negative for a disease;
assessing a performance of the classification model to produce a classification model performance assessment measure; and,
determining whether the set of ranked peptides comprises candidate biomarkers for detecting a presence of the disease in test subjects based on the classification model performance assessment measure.

24. (canceled)

25. (canceled)


SEQUENCE LISTING (129 sequences)

SEQ ID NO: 1 - protein, 8 aa, synthetic construct
  REGION 1..8: note = Synthetic
KQKGSESE

SEQ ID NO: 2 - protein, 9 aa, synthetic construct
  REGION 1..9: note = Synthetic
QKNYYWKED

SEQ ID NO: 3 - protein, 10 aa, synthetic construct
  REGION 1..10: note = Synthetic
YHVKPAVVEG

SEQ ID NO: 4 - protein, 8 aa, synthetic construct
  REGION 1..8: note = Synthetic
AARGFRED

SEQ ID NO: 5 - protein, 8 aa, synthetic construct
  REGION 1..8: note = Synthetic
PPKKVPDG

SEQ ID NO: 6 - protein, 10 aa, synthetic construct
  REGION 1..10: note = Synthetic
RLYWGSERSE

SEQ ID NO: 7 - protein, 9 aa, synthetic construct
  REGION 1..9: note = Synthetic
AARASEVSE

SEQ ID NO: 8 - protein, 9 aa, synthetic construct
  REGION 1..9: note = Synthetic
RFSQAQQSE

SEQ ID NO: 9 - protein, 8 aa, synthetic construct
  REGION 1..8: note = Synthetic
AAKGVRDG

SEQ ID NO: 10 - protein, 7 aa, synthetic construct
  REGION 1..7: note = Synthetic
KQQASDG

SEQ ID NO: 11 - protein, 9 aa, synthetic construct
  REGION 1..9: note = Synthetic
FDVSQFNSD

SEQ ID NO: 12 - protein, 8 aa, synthetic construct
  REGION 1..8: note = Synthetic
KDRGEPSE

SEQ ID NO: 13 - protein, 12 aa, synthetic construct
  REGION 1..12: note = Synthetic
KLSPQEGAPRSG

SEQ ID NO: 14 - protein, 9 aa, synthetic construct
  REGION 1..9: note = Synthetic
KDKAANRFE

SEQ ID NO: 15 - protein, 9 aa, synthetic construct
  REGION 1..9: note = Synthetic
DDKHKRFSE

SEQ ID NO: 16 - protein, 10 aa, synthetic construct
  REGION 1..10: note = Synthetic
KLKLGQVPEG

SEQ ID NO: 17 - protein, 9 aa, synthetic construct
  REGION 1..9: note = Synthetic
HGERAKVFE

SEQ ID NO: 18 - protein, 10 aa, synthetic construct
  REGION 1..10: note = Synthetic
KFGVLAEPAE

SEQ ID NO: 19 - protein, 9 aa, synthetic construct
  REGION 1..9: note = Synthetic
KYNFEERLG

SEQ ID NO: 20 - protein, 9 aa, synthetic construct
  REGION 1..9: note = Synthetic
RQKYDAQEG

SEQ ID NO: 21 - protein, 10 aa, synthetic construct
  REGION 1..10: note = Synthetic
QHVNAKAKVE

SEQ ID NO: 22 - protein, 9 aa, synthetic construct
  REGION 1..9: note = Synthetic
YRYFWKKSE

SEQ ID NO: 23 - protein, 10 aa, synthetic construct
  REGION 1..10: note = Synthetic
DNKDGRGAVE

SEQ ID NO: 24 - protein, 11 aa, synthetic construct
  REGION 1..11: note = Synthetic
NYVDHVLNHEG

SEQ ID NO: 25 - protein, 9 aa, synthetic construct
  REGION 1..9: note = Synthetic
DHKENVSEG

SEQ ID NO: 26 - protein, 10 aa, synthetic construct
  REGION 1..10: note = Synthetic
DNKDKFDPVG

SEQ ID NO: 27 - protein, 11 aa, synthetic construct
  REGION 1..11: note = Synthetic
QKKHLFPRPAD

SEQ ID NO: 28 - protein, 7 aa, synthetic construct
  REGION 1..7: note = Synthetic
DKKLNSG

SEQ ID NO: 29 - protein, 9 aa, synthetic construct
  REGION 1..9: note = Synthetic
KFSLNAKFG

SEQ ID NO: 30 - protein, 9 aa, synthetic construct
  REGION 1..9: note = Synthetic
LWNKKPAKD

SEQ ID NO: 31 - protein, 9 aa, synthetic construct
  REGION 1..9: note = Synthetic
AARQREVSE

SEQ ID NO: 32 - protein, 7 aa, synthetic construct
  REGION 1..7: note = Synthetic
HRREKDG

SEQ ID NO: 33 - protein, 11 aa, synthetic construct
  REGION 1..11: note = Synthetic
LYKSGNKYNVD

SEQ ID NO: 34 - protein, 9 aa, synthetic construct
  REGION 1..9: note = Synthetic
ELHFYNKDG

SEQ ID NO: 35 - protein, 10 aa, synthetic construct
  REGION 1..10: note = Synthetic
RREPNLDYLS

SEQ ID NO: 36 - protein, 10 aa, Borreliella burgdorferi
  REGION 1..10: note = misc_feature - O51239, Flagellar motor switch protein
IIYRKNEEFI

SEQ ID NO: 37 - protein, 10 aa, Borreliella burgdorferi
  REGION 1..10: note = misc_feature - P94284, Thioredoxin reductase
IFNKKDNVVY

SEQ ID NO: 38 - protein, 10 aa, Borreliella burgdorferi
  REGION 1..10: note = misc_feature - O51598, Uncharacterized protein
KKFIIDHTKE

SEQ ID NO: 39 - protein, 10 aa, Borreliella burgdorferi
  REGION 1..10: note = misc_feature - Q9RZW8, Adenine specific DNA methyltransferase
IKLIKDIHKD

SEQ ID NO: 40 - protein, 10 aa, Borreliella burgdorferi
  REGION 1..10: note = misc_feature - Q9S0D8, Uncharacterized protein
KNFIKDVLKD

SEQ ID NO: 41 - protein, 12 aa, Borreliella burgdorferi
  REGION 1..12: note = misc_feature - O51345, Glutamate--tRNA ligase
LLPFFQKKGYVS

SEQ ID NO: 42 - protein, 12 aa, Borreliella burgdorferi
  REGION 1..12: note = misc_feature - O51267, Leucine--tRNA ligase
NKFFDKKDNKEV

SEQ ID NO: 43 - protein, 12 aa, Borreliella burgdorferi
  REGION 1..12: note = misc_feature - O51161, Penicillin-binding protein
AKKYFKENNPES

SEQ ID NO: 44 - protein, 12 aa, Borreliella burgdorferi
  REGION 1..12: note = misc_feature - O50742, Putative phage terminase
KKYNIKFKIDEE

SEQ ID NO: 45 - protein, 10 aa, Borreliella burgdorferi
  REGION 1..10: note = misc_feature - O51731, Thymidine kinase
KIFFSKEGKE

SEQ ID NO: 46 - protein, 10 aa, Borreliella burgdorferi
  REGION 1..10: note = misc_feature - O51410, Lipopolysaccharide biosynt.-relat. protein
KYFIKPDKMM

SEQ ID NO: 47 - protein, 10 aa, Borreliella burgdorferi
  REGION 1..10: note = misc_feature - O51287, Uncharacterized protein
KIFLKDEKSV

SEQ ID NO: 48 - protein, 10 aa, Borreliella burgdorferi
  REGION 1..10: note = misc_feature - O51238, Alanine--tRNA ligase
DFIKKELSKE

SEQ ID NO: 49 - protein, 10 aa, Borreliella burgdorferi
  REGION 1..10: note = misc_feature - O51416, Lipoprotein, putative
WKKDIENKIS

SEQ ID NO: 50 - protein, 10 aa, Borreliella burgdorferi
  REGION 1..10: note = misc_feature - O51720, Uncharacterized protein
KPFFKDLNKE

SEQ ID NO: 51 - protein, 10 aa, Borreliella burgdorferi
  REGION 1..10: note = misc_feature - O51267, Leucine--tRNA ligase
FFDKKDNKEV

SEQ ID NO: 52 - protein, 10 aa, Borreliella burgdorferi
  REGION 1..10: note = misc_feature - O51498, DNA polymerase I
ASKLFKIEEK

SEQ ID NO: 53 - protein, 10 aa, Borreliella burgdorferi
  REGION 1..10: note = misc_feature - O50870, Virulent strain-associated repetitive antigen A (VraA protein)
KTEEELKKKQ

SEQ ID NO: 54 - protein, 10 aa, Borreliella burgdorferi
  REGION 1..10: note = misc_feature - O51765, NAGPA domain-containing protein
MKIENKDLKF

SEQ ID NO: 55 - protein, 10 aa, Borreliella burgdorferi
  REGION 1..10: note = misc_feature - O51765, NAGPA domain-containing protein
KIENKDLKFT

SEQ ID NO: 56 - protein, 10 aa, Borreliella burgdorferi
  REGION 1..10: note = misc_feature - O51173, Flagellar hook-associated protein 2
NKVEDYKKKY

SEQ ID NO: 57 - protein, 10 aa, Borreliella burgdorferi
  REGION 1..10: note = misc_feature - O51153, Rib
IIKGKVLVEL

SEQ ID NO: 58 - protein, 10 aa, Borreliella burgdorferi
  REGION 1..10: note = misc_feature - O51161, Penicillin-binding protein
KGENIKEFDK

SEQ ID NO: 59 - protein, 10 aa, Borreliella burgdorferi
  REGION 1..10: note = misc_feature - O51138, Replicative DNA helicase
KKEANFGIPS

SEQ ID NO: 60 - protein, 10 aa, Borreliella burgdorferi
  REGION 1..10: note = misc_feature - O51138, Replicative DNA helicase
ASISKSLKEL

SEQ ID NO: 61 - protein, 10 aa, Borreliella burgdorferi
  REGION 1..10: note = misc_feature - O51552, DNA helicase
AANEMKDRIK

SEQ ID NO: 62 - protein, 10 aa, Borreliella burgdorferi
  REGION 1..10: note = misc_feature - P70837, Uncharacterized lipoprotein BBD10
TEKNIKVEIY

SEQ ID NO: 63 - protein, 10 aa, Borreliella burgdorferi
  REGION 1..10: note = misc_feature - O51568, Transcription-repair-coupling factor
AGKNIIDPKR

SEQ ID NO: 64 - protein, 10 aa, Borreliella burgdorferi
  REGION 1..10: note = misc_feature - O50966, BBC01
DKDNIIKILK

SEQ ID NO: 65 - protein, 10 aa, Borreliella burgdorferi
  REGION 1..10: note = misc_feature - O51486, Zinc protease, putative
DKIKEVKVSL

SEQ ID NO: 66 - protein, 13 aa, Borreliella burgdorferi
  REGION 1..13: note = misc_feature - O51486, Zinc protease, putative
GKFFKKSLDDKDI

SEQ ID NO: 67 - protein, 10 aa, Borreliella burgdorferi
  REGION 1..10: note = misc_feature - O51099, Membrane protein, putative
DKIKALFISS

SEQ ID NO: 68 - protein, 10 aa, Borreliella burgdorferi
  REGION 1..10: note = misc_feature - O50957, Complement regulator-acquiring surface protein 1 (CRASP-1)
MQIKSNLEIK

SEQ ID NO: 69 - protein, 10 aa, Borreliella burgdorferi
  REGION 1..10: note = misc_feature - Q9S093, Uncharacterized protein
IKGAFDGIKN

SEQ ID NO: 70 - protein, 10 aa, Borreliella burgdorferi
  REGION 1..10: note = misc_feature - O51544, UDP-N-acetylenolpyruvoylglucosamine reductase
CKEFKKEDFK

SEQ ID NO: 71 - protein, 10 aa, Borreliella burgdorferi
  REGION 1..10: note = misc_feature - O50692, Putative lipoprotein
KDFIKNSEKL

SEQ ID NO: 72 - protein, 11 aa, Borreliella burgdorferi
HSLDQVLQKRG

SEQ ID NO: 73 - protein, 11 aa, Borreliella burgdorferi
KQEGAQLPNRE

SEQ ID NO: 74 - protein, 8 aa, Borreliella burgdorferi
DKDFANKE

SEQ ID NO: 75 - protein, 8 aa, Borreliella burgdorferi
PKKANASD

SEQ ID NO: 76 - protein, 8 aa, Borreliella burgdorferi
QEGKQEAG

SEQ ID NO: 77 - protein, 10 aa, Borreliella burgdorferi
PWKSFFPQED

SEQ ID NO: 78 - protein, 9 aa, Borreliella burgdorferi
PKGGNAKVS

SEQ ID NO: 79 - protein, 9 aa, Borreliella burgdorferi
HQEGASGDG

SEQ ID NO: 80 - protein, 8 aa, Borreliella burgdorferi
VKKFAQKD

SEQ ID NO: 81 - protein, 9 aa, Borreliella burgdorferi
SQLAKSDDG

SEQ ID NO: 82 - protein, 9 aa, Borreliella burgdorferi
QRYLRGRLS

SEQ ID NO: 83 - protein, 8 aa, Borreliella burgdorferi
FEVFFDLD

SEQ ID NO: 84 - protein, 9 aa, Borreliella burgdorferi
LSRSRGYFG

SEQ ID NO: 85 - protein, 9 aa, Borreliella burgdorferi
FYFLFAKSD

SEQ ID NO: 86 - protein, 10 aa, Borreliella burgdorferi
YSASRLYFSG

SEQ ID NO: 87 - protein, 11 aa, Borreliella burgdorferi
YRPQRSLSYFS

SEQ ID NO: 88 - protein, 9 aa, Borreliella burgdorferi
QDAAEWKLE

SEQ ID NO: 89 - protein, 11 aa, Borreliella burgdorferi
YGQEGGWNQRE

SEQ ID NO: 90 - protein, 8 aa, Borreliella burgdorferi
FKFLRLFG

SEQ ID NO: 91 - protein, 10 aa, Borreliella burgdorferi
YRALNQFRLF

SEQ ID NO: 92 - protein, 9 aa, Borreliella burgdorferi
VGAFALVFG

SEQ ID NO: 93 - protein, 9 aa, Borreliella burgdorferi
LQLYLEDFD

SEQ ID NO: 94 - protein, 9 aa, Borreliella burgdorferi
FYDYLFEPD

SEQ ID NO: 95 - protein, 7 aa, Borreliella burgdorferi
FFLDFED

SEQ ID NO: 96 - protein, 7 aa, Borreliella burgdorferi
LFFVDEG

SEQ ID NO: 97 - protein, 10 aa, Borreliella burgdorferi
PDYLFDFLFE

SEQ ID NO: 98 - protein, 7 aa, Borreliella burgdorferi
AFLLLSE

SEQ ID NO: 99 - protein, 10 aa, Borreliella burgdorferi
YFYFDPYPVG

SEQ ID NO: 100 - protein, 10 aa, Borreliella burgdorferi
VFFEFDNVLE

SEQ ID NO: 101 - protein, 10 aa, Borreliella burgdorferi
RSYAGYRQLS

SEQ ID NO: 102 - protein, 10 aa, Borreliella burgdorferi
YRALNQFRLF

SEQ ID NO: 103 - protein, 8 aa, Borreliella burgdorferi
FFFEQVED

SEQ ID NO: 104 - protein, 10 aa, Borreliella burgdorferi
SYRREDKFEG

SEQ ID NO: 105 - protein, 10 aa, Borreliella burgdorferi
FLAFSEPYED

SEQ ID NO: 106 - protein, 10 aa, Borreliella burgdorferi
  REGION 1..10: note = misc_feature - P0CL66, Outer surface protein A (OspA)
TKENTITVQQ

SEQ ID NO: 107 - protein, 13 aa, Borreliella burgdorferi
  REGION 1..13: note = misc_feature - O50927, Bacterial extracellular solute-binding protein
IKYNKNDLEKEVA

SEQ ID NO: 108 - protein, 10 aa, Borreliella burgdorferi
  REGION 1..10: note = misc_feature - H7C7N8, Integral outer membrane protein p66
DNKKGIINGL

SEQ ID NO: 109 - protein, 10 aa, synthetic construct
  REGION 1..10: note = Synthetic
KQWNCNDKKC

SEQ ID NO: 110 - protein, 10 aa, synthetic construct
  REGION 1..10: note = Synthetic
KIWYMNEKGE

SEQ ID NO: 111 - protein, 13 aa, Borreliella burgdorferi
  REGION 1..13: note = misc_feature - O51633, Neutrophil activating protein A (NapA)
KKSFIKELDIEST

SEQ ID NO: 112 - protein, 11 aa, Borreliella burgdorferi
  REGION 1..11: note = misc_feature - O51069, Chemotaxis protein methyltransferase CheR
KKLNLMDEKFP

SEQ ID NO: 113 - protein, 13 aa, Borreliella burgdorferi
  REGION 1..13: note = misc_feature - G5IXI6, Variable large protein VlsE
KFAVKDGEKEKAE

SEQ ID NO: 114 - protein, 10 aa, synthetic construct
  REGION 1..10: note = Synthetic
HMACNWKEPL

SEQ ID NO: 115 - protein, 13 aa, Borreliella burgdorferi
  REGION 1..13: note = misc_feature - O51677, Probable endolytic peptidoglycan transglycosylase RlpA
KTTANGEKFDMMA

SEQ ID NO: 116 - protein, 12 aa, Borreliella burgdorferi
  REGION 1..12: note = misc_feature - O51715, Flagellar basal-body rod protein FlgG (Distal rod protein)
TGFKKIRAEFED

SEQ ID NO: 117 - protein, 10 aa, synthetic construct
  REGION 1..10: note = Synthetic
HQTKQFKCVE

SEQ ID NO: 118 - protein, 13 aa, Borreliella burgdorferi
  REGION 1..13: note = misc_feature - O51778, LptD Lipopolysaccaride assembly membrane protein
KKWALKEGLKETD

SEQ ID NO: 119 - protein, 10 aa, synthetic construct
  REGION 1..10: note = Synthetic
KNTYFQEHKD

SEQ ID NO: 120 - protein, 10 aa, Borreliella burgdorferi
  REGION 1..10: note = misc_feature - P11089, Flagellar filament 41 kDa core protein (Flagellin) FlaB
EGVQQEGAQQ

SEQ ID NO: 121 - protein, 10 aa, Borreliella burgdorferi
  REGION 1..10: note = misc_feature - Q45013, Borrelia P83/P100 antigen
INLDKAQQKL

SEQ ID NO: 122 - protein, 14 aa, Borreliella burgdorferi
  REGION 1..14: note = misc_feature - O51267, Leucine--tRNA ligase (Leucyl-tRNA synthetase) LeuRS
NKFFDKKDNKEVTQ

SEQ ID NO: 123 - protein, 13 aa, Borreliella burgdorferi
  REGION 1..13: note = misc_feature - O51498, DNA polymerase I (PolA)
IFTKKLKEDKLDK

SEQ ID NO: 124 - protein, 13 aa, Borreliella burgdorferi
  REGION 1..13: note = misc_feature - O50725, Putative surface protein, Involved in colonization BBE31
KKYEKKLEEEPSN

SEQ ID NO: 125 - protein, 11 aa, Borreliella burgdorferi
  REGION 1..11: note = misc_feature - O50743, Putative phage portal protein, HI1409 family
HKMMILGREDD

SEQ ID NO: 126 - protein, 10 aa, Borreliella burgdorferi
  REGION 1..10: note = misc_feature - C6 peptide of the VlsE protein
GKFAVKDGEK

SEQ ID NO: 127 - protein, 8 aa, synthetic construct
  REGION 1..8: note = Synthetic
KPLEEVLN

SEQ ID NO: 128 - protein, 7 aa, synthetic construct
  REGION 1..7: note = Synthetic
FLPFQQK

SEQ ID NO: 129 - protein, 13 aa, Borreliella burgdorferi
  REGION 1..13: note = misc_feature - Q9S035, ErpQ protein
CKNFATGKDIKQN